(19) United States
(12) Patent Application Publication
(10) Pub. No.: US 2021/0313026 A1
(43) Pub. Date: Oct. 7, 2021

(54) SYSTEMS AND METHODS FOR ACCELERATED EPIDEMIC RECOVERY

(71) Applicant: BuddyCheque, LLC, Reno, CA (US)

(72) Inventors: Karl Theodore Wagner, Reno, NV (US); Alexandre Chatel, Savoie (FR); Ramsey Kilani, Raleigh, NC (US); Robert Eugene Ramsey, Tempe, AZ (US)

(21) Appl. No.: 16/900,846

(22) Filed: Jun. 12, 2020

Related U.S. Application Data
(60) Provisional application No. 63/004,418, filed on Apr. 2, 2020.
(60) Provisional application No. 63/020,508, filed on May 5, 2020.

(51) Int. Cl. G16H 10/65; G16H 10/40; G16H 50/70; G16H 50/20; G16H 50/30; G16H 50/80; G06Q 50/26; G06F 21/60; H04L 9/08; A61B 5/1171; A61B 5/00

(52) U.S. Cl. CPC G16H 10/65; G16H 10/40; G16H 50/70; G16H 50/20; G16H 50/30; A61B 5/6898; G06Q 50/265; G06F 21/602; H04L 9/083; A61B 5/1176; A61B 5/0077; G16H 50/80

(57) ABSTRACT

A method comprising receiving, from a user device, a user identifier, the user identifier being associated with a user of the user device, confirming that the user's identity has been confirmed, retrieving, from a remote health record maintained by a third-party health service provider, health information regarding a virus-related test provided on the user, providing a virus-related test status based on the virus-related test results to the user device, receiving an invalidity indication from the third-party health-related entity, the invalidity indication indicating that a particular test is no longer considered valid, reviewing a record of the user to confirm if the user received the particular test, determining that the virus-related test is no longer valid based on the invalidity indication, providing a notice to the user device that the virus-related test status is no longer considered to be valid, and providing an update to the virus-related test status.

### CROSS-REFERENCE FIELD

This application receives the benefit of U.S. provisional patent application Ser. No. 63/004,418, filed Apr. 2, 2020, titled “Systems and Methods for Accelerating COVID-19 Recovery” and U.S. provisional patent application Ser. No. 63/020,508, filed May 5, 2020, titled “Systems and Methods for Accelerating COVID-19 Recovery,” both of which are incorporated by reference herein.

### TECHNICAL FIELD

This disclosure pertains to systems for dissemination and confirmation of health information across different patients of different health systems to enable opportunity and, more specifically, a centralized architecture for confirmation and dissemination of identity and testing for illnesses for individuals and those who need to assess proximity-based risk in view of contagion.

### BACKGROUND

Virus epidemics and even pandemics are inevitable. Even as modern science improves, there are still a number of viruses that continue to be extremely contagious, potentially life threatening (e.g., to those with weakened immune systems), and incurable. The question is not “if” an epidemic or pandemic will occur, but rather “when.”

Medical science will continue to strive against these illnesses and viruses as they evolve. The process of finding cures, producing vaccinations, and treating the sick is still time-consuming and can bring economies to a halt because of shelter-in-place requirements to control the virus and other controls.

In the case of COVID-19, many people throughout the world and the US are infected and have been infected. In an effort to reduce the number of new cases to better match medical resources, many governments have attempted to reduce the opportunity of the virus to spread by ordering all but essential businesses to halt. These orders further mandate that people stay home and/or take other precautions to reduce possible exposure.

### SUMMARY

An example nontransitory computer readable medium comprises instructions that are executable by a processor. The instructions being executable to perform an example method. The method comprising receiving, from a user device, a user identifier, the user identifier being associated with a user of the user device, confirming that the user's identity has been confirmed, retrieving, from a remote health record maintained by a third-party health service provider, health information regarding a virus-related test provided on the user, the virus-related test including a test identifier, a date when the virus-related test was given, and virus-related test results, providing a virus-related test status based on the virus-related test results to the user device, if the virus-related test is determined to be invalid, receiving an invalidity indication from a third-party health-related entity, the invalidity indication indicating that a particular test is no longer considered valid, reviewing a record of the user of the user device to confirm if the user received the particular test, determining that the virus-related test is no longer valid based on the invalidity indication, providing a notice to the user device that the virus-related test status is no longer considered to be valid, and providing an update to the virus-related test status based on the invalidity indication.

In various embodiments, the virus-related test is an infection test and the virus-related test status is an indication if the user is infected. The virus-related test is an immunity test and the virus-related test status may be an indication if the user is immune.

In some embodiments, the method may further comprises receiving, from an other user device, an other user identifier, the user device and the other user device being remote from each other, confirming that the other user's identity has been confirmed, retrieving, from an other remote health record maintained by an other third-party health service provider, health information regarding an other virus-related test provided on the other user, the other virus-related test including an other test identifier, an other date when the other virus-related test was given, and other virus-related test results, the third-party health service provider and the other third-party health service provider being remote to each other and being operated by different entities, and providing an other virus-related test status based on the other virus-related test results to the other user device.

Receiving, from the user device, may comprise receiving a user image from the user device. The method further comprising receiving information regarding a trusted image of the user and performing identity confirmation based on the user image and the information regarding the trusted image to confirm the user's identity. Retrieving, from the remote health record maintained by the third-party health service provider, health information regarding the virus-related test provided on the user may comprise retrieving an encryption key associated with the user and providing a request for the health information with the encryption key to the third-party health service provider. The method may further comprise receiving a virus-related test indication associated with the user, the virus-related test indication indicating the third-party health service provider as having the health information regarding the virus-related test provided on the user. Retrieving, from the remote health record maintained by the third-party health service provider, health information regarding the virus-related test provided on the user may comprise providing a request for the health information using an identifier that does not refer to the user.

In some embodiments, retrieving, from the remote health record maintained by the third-party health service provider, health information regarding the virus-related test provided on the user comprises receiving, without providing an initial request, the health information regarding the virus-related test from the third-party health service provider, storing the health information regarding the virus-related test, and retrieving the health information regarding the virus-related test from storage. The method may further comprise receiving, from the user device, the user identifier, confirming that the user's identity has been confirmed, retrieving, from a remote health record maintained by a third-party health service provider, health information regarding a vaccination provided to the user, and providing an immunity classification to the user device, the immunity classification indicating that the user is immune to at least one illness. In some embodiments, the method further comprise receiving, from the user device, the user identifier, confirming that the user's identity has been confirmed, retrieving, from a remote health record maintained by a third-party health service provider, health information regarding an antibody test provided on the user and immunity test results, scoring the immunity test results to classify the user regarding possible immunity to infection to at least on illness, and providing a immunity classification based on the scoring to the user device, the immunity classification indicating whether the user is immune or likely immune.

An example method may comprise receiving, from a user device, a user identifier, the user identifier being associated with a user of the user device, confirming that the user's identity has been confirmed, retrieving, from a remote health record maintained by a third-party health service provider, health information regarding a virus-related test provided on the user, the virus-related test including a test identifier, a date when the virus-related test was given, and virus-related test results, providing a virus-related test status based on the virus-related test results to the user device, if the virus-related test is determined to be invalid, receiving an invalidity indication from a third-party health-related entity, the invalidity indication indicating that a particular test is no longer considered valid, reviewing a record of the user of the user device to confirm if the user received the particular test, determining that the virus-related test is no longer valid based on the invalidity indication, providing a notice to the user device that the virus-related test status is no longer considered to be valid, and providing an update to the virus-related test status based on the invalidity indication.

An example system may include at least one processor and memory. The memory may include instructions configured to control the at least one processor to receive, from a user device, a user identifier, the user identifier being associated with a user of the user device, confirm that the user's identity has been confirmed, retrieve, from a remote health record maintained by a third-party health service provider, health information regarding a virus-related test provided on the user, the virus-related test including a test identifier, a date when the virus-related test was given, and virus-related test results, provide a virus-related test status based on the virus-related test results to the user device, if the virus-related test is determined to be invalid, receive an invalidity indication from a third-party health-related entity, the invalidity indication indicating that a particular test is no longer considered valid, review a record of the user of the user device to confirm if the user received the particular test, determine that the virus-related test is no longer valid based on the invalidity indication, provide a notice to the user device that the virus-related test status is no longer considered to be valid, and provide an update to the virus-related test status based on the invalidity indication.

### BRIEF DESCRIPTION OF THE DRAWINGS

FIG. 1 depicts an example health interface in some embodiments.

FIG. 2 depicts an example environment for verifying and disseminating information in some embodiments.

FIG. 3 is a block diagram depicting a user smart device in some embodiments.

FIG. 4 is a block diagram depicting a TIVD system in some embodiments.

FIG. 5 depicts an example flowchart of confirming a user's identity in some embodiments.

FIG. 6 is a flowchart for receiving and verifying infection test information in some embodiments.

FIG. 7 is a flowchart for receiving and verifying immunity information in some embodiments.

FIG. 8 is a flow diagram of calculating a score based on test measurements in some embodiments.

FIG. 9 is an example method of providing an infection summary and/or an immunity summary to a venue employee in some embodiments.

FIG. 10 is a method for tracking test results and classifications such that summaries may be controlled or otherwise indicated to be expired or no longer valid.

FIG. 11A depicts an example interface indicating that the user's identity is unverified and the user has not been tested for infection or immunity.

FIG. 11B depicts an example interface indicating that the user's identity is verified and the user has not been tested for infection or immunity.

FIG. 11C depicts an example interface indicating that the user's identity is verified and the user has been tested for infection and immunity.

FIG. 11D depicts an example interface indicating that the user's identity is verified and the user has been tested for infection but not immunity.

FIG. 11E depicts an example interface indicating that the user's identity is verified and the user has not been tested for infection but has been tested for immunity.

FIG. 12A depicts an example interface indicating that the user's identity is verified and the user has been tested for infection and immunity.

FIG. 12B depicts an example interface indicating that the user's identity is verified and the user has been tested for infection and immunity.

FIG. 12C depicts an example interface indicating that the user's identity is verified and the user has been tested for infection and immunity.

FIG. 13 depicts a block diagram of an example digital device according to some embodiments

### DETAILED DESCRIPTION

When there is an ongoing epidemic or pandemic, such as the COVID-19 pandemic, there is an ongoing dilemma between (1) limiting opportunities for the virus to spread and (2) avoiding a profound and sustained economic downturn that threatens the ability of people to be employed, purchase essentials, and take care of their families. In order to avoid the economic downturn, businesses stay open and people must go to work. However, opening businesses and return to work causes increased opportunities for the spread of viruses. If businesses reopen too soon, the number of infections may skyrocket and many may die because medical resources are overwhelmed. If businesses do not reopen, then many people (particularly the middle and poorer classes) and their families broadly suffer. Further, other people suffer because products and services are not being purchased.

It is important to note that testing typically becomes available far before a solution, cure, or vaccine is developed. There are generally two types of tests: (1) a test that indicates whether a person being tested has the virus (or a mutation of it); and (2) a test that indicates if the person has had the virus and, as such, may be at least partially immune (or immune for a while). It may be appreciated that when a person recovers from infection of many viruses, the person may have some immunity against reinfection (e.g., over a limited period of time or a decreased risk of infection). Other viruses, however, may leave a formerly infected person with no immunity.

As vaccinations become available, there will be those who are immune due to vaccinations. As time progresses, people may be tested to determine if they have immunity due to vaccinations, immunity due to past infection, and/or the like.

One method to navigate the dilemma between opening businesses and limiting the opportunities for the virus to spread is to have a trusted system that will confirm and/or disseminate information including identification of people and identification of their health status regarding an epidemic/pandemic. Various embodiments described herein include a solution for empowerment of citizens, prompt economic recovery, and responsible governance of sensitive population health data. The examples shown herein depict embodiments as they relate to the COVID-19 epidemic/pandemic. It may be appreciated that systems and methods described herein may be used in relation to any health condition or combination of health conditions that may be tested. Further, although information regarding immunity and testing is discussed as being disseminated using the system and methods discussed herein, it will be appreciated that these systems and methods are not limited to immunity and testing, but may apply to non-health care industries such as testing and treatment of food (e.g., produce or livestock), testing of chemicals, location of equipment or assets, and/or the like.

Many of the figures described or herein describe a system that may enable communities, businesses, and governments to know and share certified COVID-19 immunity status information. The system may, in some embodiments, be a reliable re-employment platform.

It will be appreciated that actionable data collection and systematic sharing may be critical in reopening the economy while still limiting people's exposure to COVID-19. In some embodiments, businesses could limit in-person employee and customer interaction to those that are known (or very likely) not infected or immune. Immune employees as well as those employees that are not infected may be allowed to go to work. Customers that are immune or are known (or very likely) not infected, may be able to go to businesses and/or travel (e.g., by plane).

A trusted secure system is described in some embodiments that, at scale, identifies each person and provides their immunity/infection status to allow for economic recovery, families to come together, and society to begin the healing process.

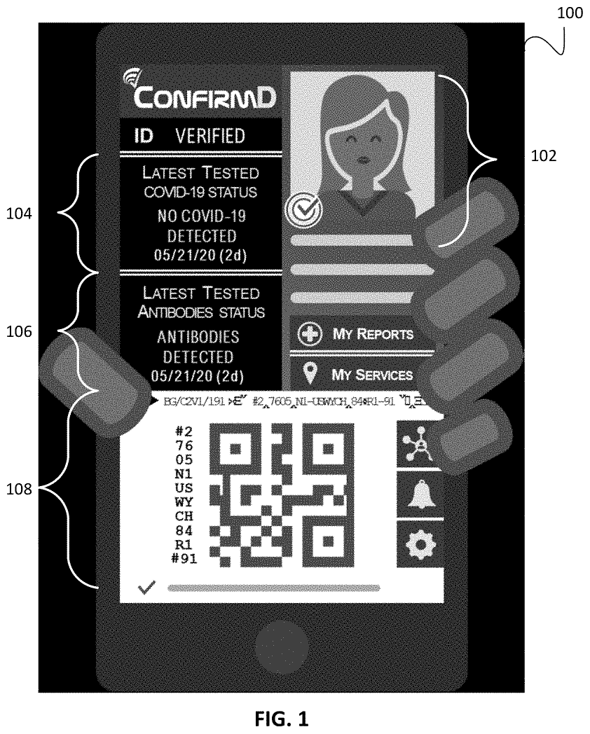

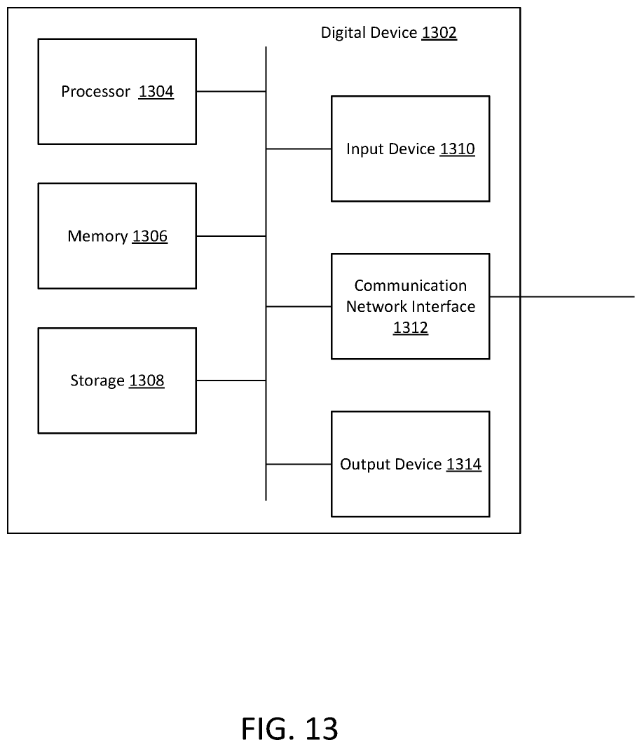

FIG. 1 depicts an example health interface 100 in some embodiments. The health interface 100 may be depicted on a user's smart device. A smart device may be any device with a processor and memory (e.g., the digital device of FIG. 13). Examples of a user's smart device may include a smart phone, media tablet, smartwatch, laptop, smart wearable, and/or the like.

In some embodiments, an application on the user's smart device may verify a user's identity and provide health information. In one example, the application on the user's smart device may, for example, scan a user's identification information from a trusted document (e.g., driver's license, passport, and/or the like). The application may also receive or take an image (e.g., an untrusted image) of the user. The application may perform facial recognition through comparison of the user's image and the user's identification information from the trusted document. In various embodiments, the application may also confirm the user's identification information (e.g., confirm the passport and/or driver's license).

The user's smart device may receive the user's health information from a cloud service, laboratory, testing service, and/or the like. Based in part on the confirmation of the user's identity, the user's smart device may display the user's information and the health information. The health information may include an image of the user, infection summary, immunity summary, and/or a code to enable other digital device to retrieve information regarding the user and the user's status information.

In the example depicted in FIG. 1, the health interface 100 depicts a single interface or card depicted on user's smart phone. In this example, the health interface 100 includes an image 102 of the user, infection summary information 104, immunity summary information 106, and health code 108. It will be appreciated that, in some embodiments, the heath interface 100 is designed for easy communication of a user's health condition relative to one or more diseases or viruses. For example, the health information 100 may include information of the user's identity, an indication that the identity was confirmed, a quick indication of a user's most recent test results for illness, a quick indication of a user's possible immunity (e.g., most recent test results/vaccination date), and, optionally a code to enable other devices to confirm the information with a centralized system.

In some embodiments, the health interface may not include the immunity summary information 106 (e.g., there are no known methods to obtain immunity to a particular virus).

The image 102 of the user may be an image taken from the trusted document. A trusted document may, for example, be a government issued document such as a driver's license, real identification card, passport, Certificate of Naturalization, Certificate of Citizenship, Government Employee ID, US Military ID and Military Dependent ID, US permanent resident card, trusted traveler IDs, enhanced travel cards Native American tribal photo IDs and/or the like. Alternately, the image 102 of the user may be an image of the user taken by the smart device (e.g., via a camera) or may be an image of the user selected by the user (e.g., from the user's photographs on the phone).

The health interface may also indicate if the user's identification has been verified. The user's identification may be verified in any number of ways. For example, the user's identification may be verified by the smart device (or a cloud device) comparing the user's image to a government issued document or other information obtained from the user or other sources (e.g., from a government database, or service).

The infection summary information 104 may include information regarding the user's health testing. Health testing information may be or include a summary of information regarding the user's recent testing for a particular disease (e.g., virus). In this example, infection summary information 104 is depicted partially adjacent to the image 102 of the user. The infection summary information 104 may indicate the last testing on the user, including what was tested (e.g., COVID-19), results of the test (e.g., No COVID-19 detected), and date of the test (e.g., May 21, 2020).

The health interface 100 may enable the user or someone else to interact with the infection summary information 104 (e.g., by clicking or touching that part of the interface). When engaged, the health interface 100 may provide additional information regarding the infection summary information 104 such as the location of the testing, laboratory that performed the test, test results, and/or the like.

The immunity summary information 106 may include information regarding the user's existing or possible immunity. Immunity testing information may be or include a summary of information regarding the user's recent testing for antibodies (e.g., an indication that the user has recovered from a particular disease). In that example, the immunity testing information may include the most recent date of immunity testing. The immunity summary information 106 may include an indication that the user has been vaccinated as well as the date of vaccination.

The health interface 100 may enable the user or someone else to interact with the immunity summary information 106 (e.g., by clicking or touching that part of the interface). When engaged, the health interface 100 may provide additional information regarding the immunity summary information 106 such as the location of the testing, laboratory that performed the test, test results, and/or the like. In some embodiments, the health interface 100 may provide additional information regarding the immunity summary information 106 such as the location of the vaccination, facility that performed the vaccination, the type and delivery of the vaccination, and/or the like.

In this example, immunity summary information 106 is depicted below the infection summary information 104 in the health interface 100. The immunity summary information 106 may indicate the last testing on the user, including what was tested (e.g., antibodies for COVID-19), results of the test (e.g., antibodies for COVID-10 detected), and date of the test (e.g., May 21, 2020).

The health information may include an image of the user, infection summary, immunity summary, and/or a code to enable other digital device to retrieve information regarding the user and the user's status information.

It will be appreciated that the health interface 100 may include any amount of information in not be limited to the image of the user, infection summary, immunity summary, and/or codes. Further, there may be any number of health interfaces that display any or all of this information in any order, orientation, or position.

In some embodiments, the health interface 100 may have options to transition between interfaces to display testing and/or immunity information regarding different diseases. For example, someone may be able to press icons or swipe left/right to depict new infection summary information and/or immunity summary information regarding different diseases or medical conditions.

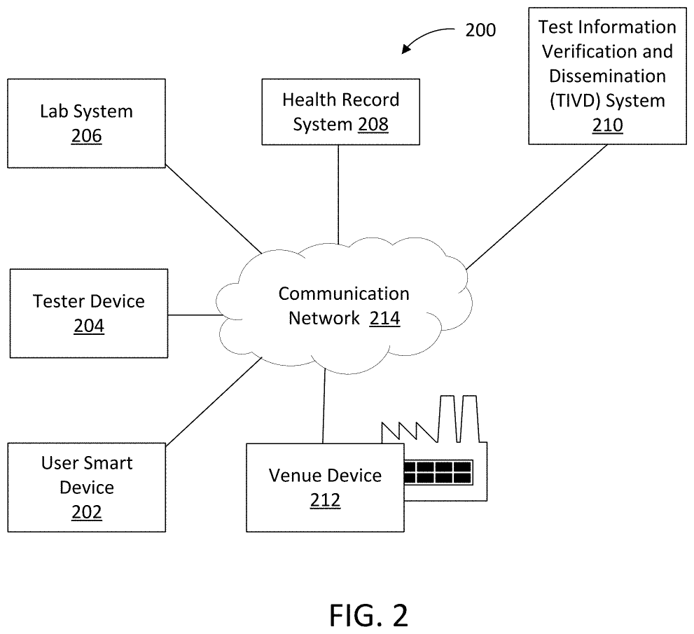

FIG. 2 depicts an example environment 200 for verifying and disseminating information in some embodiments. The environment 200 includes a user smart device 202, a tester device 204, a laboratory (lab) system 206, a health record system 208, a test information verifying and dissemination (TIVD) system 210, a venue device 212, and a communication network 214.

The user smart device 202 may be the user smart device described regarding FIG. 1. The user smart device 202 may be, for example, a smart phone, media tablet, smart wearable, computer, laptop, and/or the like. The user smart device 202 may include an application that enables the user to input identification information, verify the identification information, receive testing information, receive the immunity information, display infection summary information, display immunity summary information, and/or the like. The user smart device 202 may include an application that displays the health interface 100.

The tester device 204 may be a device associated with a health care specialist. The health care specialist may test the user for illness (e.g., COVID-19). In some embodiments, the health care specialist will provide test results to a health care provider, health care service entity (e.g., laboratory), and/or provide information regarding the test (e.g., reagents used, test type used, date of test, conditions of the test, and/or the like). The health care provider may include a laboratory, insurance company, hospital, clinic, and/or the like.

In some embodiments, the health care specialist may provide testing for disease, testing for antibodies, and/or vaccinations. The health care specialist may upload information regarding the illness test, immunity test, and/or vaccination to the health care facility using the tester device 204.

In various embodiments, the tester device 204 may comprise a tester version of the application and the user smart device 202 may include a tester version of the application. The user may provide his identity to a health care specialist (e.g., person providing a test or vaccine) as normally done in a health procedure. The health care specialist may input identity information within the tester version of the application and/or a health record (e.g., maintained by the health record system 208). The health care specialist may then conduct tests and provide the results to the tester version of the application and/or a health record.

The lab system 206 may be a digital device associated with a laboratory, clinic, hospital, or the like. A digital device is any device with memory and a processor. The laboratory system may analyze test results, blood, bodily fluids, and/or the like. In various embodiments, the tester may provide samples to the laboratory for analysis. The lab system 206 may receive the results of the analysis (e.g., indicating infection, indicating antibodies, and/or the like). The lab system 206 may provide the analytical results to a health record associated with the person being tested.

The health record system 208 may include any electronic health record system maintained by, for example, an insurance company, hospital, hospital system, laboratory, third-party, clinic, health provider, and/or the like. An example health record system 208 may be or include the EPIC system.

The test information, verification, and dissemination (TIVD) system 210 may assist in verifying the identity of users, maintaining or tracking testing, testing results, disseminating results, generating categories (e.g., “infected,” “not infected,” “immune due to antibody testing,” “immune due to vaccination,” and/or the like).

The platform may aggregate certain medical test and vaccine data, including one or more of the following (for example):

COVID-19 polymerase chain reaction (PCR) test report (results usually provided by a laboratory or health service within 24 hours of receiving test)

COVID-19 IgG antibody test report (results usually within one hour)

CT Scan report (for COVID-19 diagnosis)

Vaccination records

Other clinical, paraclinical and demographic data

The test results may be data may be verified by medical professionals.

The TIVD system 210 may utilize some or all of the information to generate an immunity score. The immunity score may be calculated in any number of ways. In one example, the immunity score is a weighted sum of information such as: (PCR)(0.9)+IgG(0.9)+ct(0.8)=immunity score.

In another example, if there is only a positive PCR score, then the immunity score may be set to 90%. If there is only a positive IgG score, the immunity also be set to 90%. If there are both positive PCR and the IgG scores indicating recovery, then the immunity score may be set to 99%. Similarly, if there is only a CT score that is positive, the immunity score may be set to 80% and if there is both a CT score and a lab score, the immunity score may be set to 99%.

In another example, if IgG levels are over 200 mg/dL, then there may be a strong indication of immunity. In another example, a PCR test may be scaled on levels of (for example) 1 to 5 based on likely immunity. Scoring may be based on a combination of both tests or more (e.g., weighted based on test and/or how recent the tests are). If a user does not have a positive PCR test, then a time clock may start based on date of exposure, confirmation of antibodies.

It will be appreciated that the immunity score may be set based on recent test data from different medical and/or research facilities regarding dependability of the different testing approaches. The immunity score may be based on other test indicators as well. As additional data including scores and/or confirmation of positive or negative results, the process of scoring and determining a classification may be curated and improved (e.g., through AI model testing and improvement and/or statistical modeling).

In some embodiments, a goal of the TIVD system 210 is to calculate an immunity score to indicate a degree of confidence that the user has recovered and/or has a degree of immunity (e.g., is immune) from COVID-19. These individuals may have a degree of immunity and therefore may be encouraged to enter back into public life and work to restart and/or improve the economy and society.

The TIVD system 210 may confirm test results from different sources to prevent fraud or error. The platform may also provide calculations to confirm the test score.

In various embodiments, the TIVD system 210 may safeguard the immunity scores by storing and encrypting the information such that: (1) the platform may certify the result; and (2) only those authorized to receive the information will be provided information. In some embodiments, identify verification may be encrypted and stored in a secure location that is separate from the encrypted, secure storage that contains test results.

There may be different thresholds for different immunity scores. Different employers may decide that different immunity scores are sufficient for the employee to come back to work (e.g., jobs that involve significant contact with the public may require higher immunity scores than those that work in more solitary rolls).

In some embodiments, a user may register with the TIVD system 210 and the user's employer may register with the TIVD system 210. The user may provide the employer with permission to receive their immunity score (e.g., their immunity score or their immunity score only if it is above a certain threshold). The employer may provide sufficient evidence that they employ the user and, in some embodiments, the platform may confirm employment (e.g., through tax records, documents proving employment, and/or the like).

If the user is certified as having a high immunity score (e.g., 99%), the employer may send an authenticated request to the TIVD system 210 to receive or confirm the user's particular immunity score (or, in some embodiments, the employer may not receive the immunity score, but only an indication of immunity). Based on that information, the employer may make decisions such as, for example, allowing that particular employee to come back to work.

The venue device 212 may be any device associated with a facility such an office, stadium, restaurant, theater, airport, and the like. In one example, an office or airport may wish to station personnel at entrance points of the venue. As each person approaches the venue for entrance (e.g., to go to a game, see a performance, go to work, fly on a plane, or the like), the personnel may request the user to provide their health interface 100 such that the personnel may confirm that the person has been tested, is not infected, is immune, or has obtained a necessary criteria of safety before allowing the person entrance to the venue. In some embodiments, personnel may have their own digital device with a venue application (e.g., a venue TIVD) application that may scan the user's code from the user's health interface 100 and then confirm the health information through the TIVD system 210.

In accordance with some embodiments, the computer network 214 may be implemented or facilitated using one or more local or wide-area communications networks, such as the Internet, WiFi networks, WiMax networks, private networks, public networks, personal area networks (“PAN”), and the like. In some embodiments, the computer network 214 may be a wired network, such as a twisted pair wire system, a coaxial cable system, a fiber optic cable system, an Ethernet cable system, a wired PAN constructed with USB and/or FireWire connections, or other similar communication network. Alternatively, the computer network 214 may be a wireless network, such as a wireless personal area network, a wireless local area network, a cellular network, or other similar communication network. Depending on the embodiment, some or all of the communication connections with the computer network 214 may utilize encryption (e.g., Secure Sockets Layer [SSL]) to secure information being transferred between the various entities shown in FIG. 1.

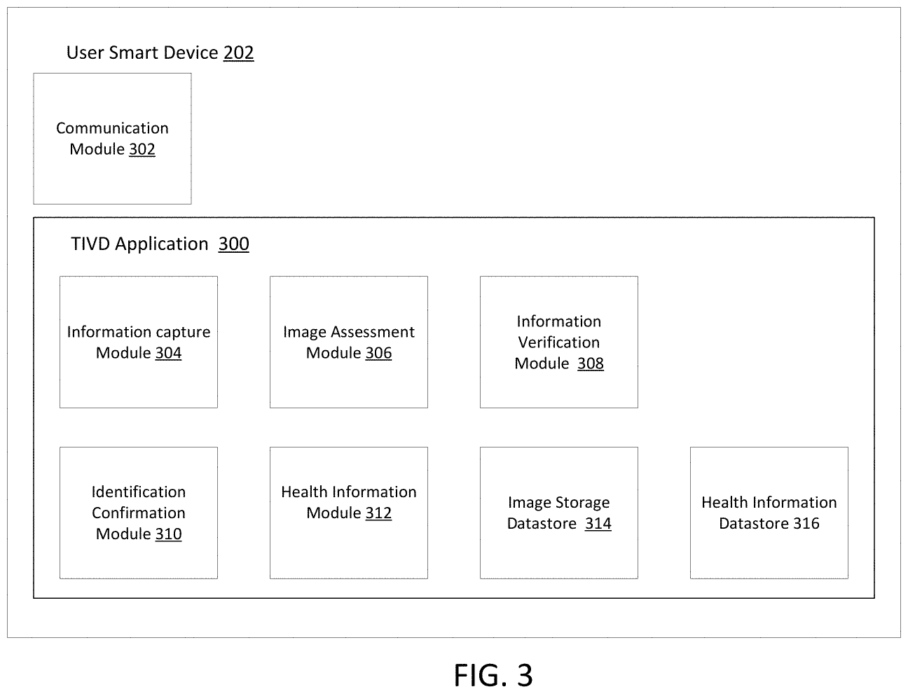

FIG. 3 is a block diagram depicting a user smart device 202 in some embodiments. User smart device 202 may include a communication module 302 and a TIVD application 300. The TIVD application 300 may include an information capture module 304, an image assessment module 306, an information verification module 308, an identification confirmation module 310, a health information module 312, an image storage datastore 314, and a health information datastore 316.

The communication module 302 may be configured to send requests to and receive data from one or a plurality of systems. The communication module 302 is configured to communicate with one or more digital devices over the communication network 214. For example, the communication module 302 may receive identity information from the use user. The communication module 302 may send requests to and receive data from a system through a network or a portion of a network. Depending upon implementation-specific or other considerations, the communication module 302 may send requests and receive data through a connection, all or a portion of which may be a wireless connection. The communication module 302 may request and receive messages, and/or other communications from associated systems.

The information capture module 304 may be configured to capture information such as an image of the user, a document (e.g., a trusted document) identifying the user, test information (e.g., infection test information, immunity test information, infection test indications, immunity indications, vaccination indications, and/or the like). In various embodiments, the information capture module 304 receives an image of the user from a camera or other sensor on the user smart device 202. Similarly, in this example, the information capture module 304 may receive an image of the document from the camera or other sensor. The information capture module 304 may receive an indication of a test that is performed on the user (e.g., infection or antibody test), an indication that the user received a vaccination, test results, information regarding any tests (e.g., type of test, name of test, illness tested for) and the like.

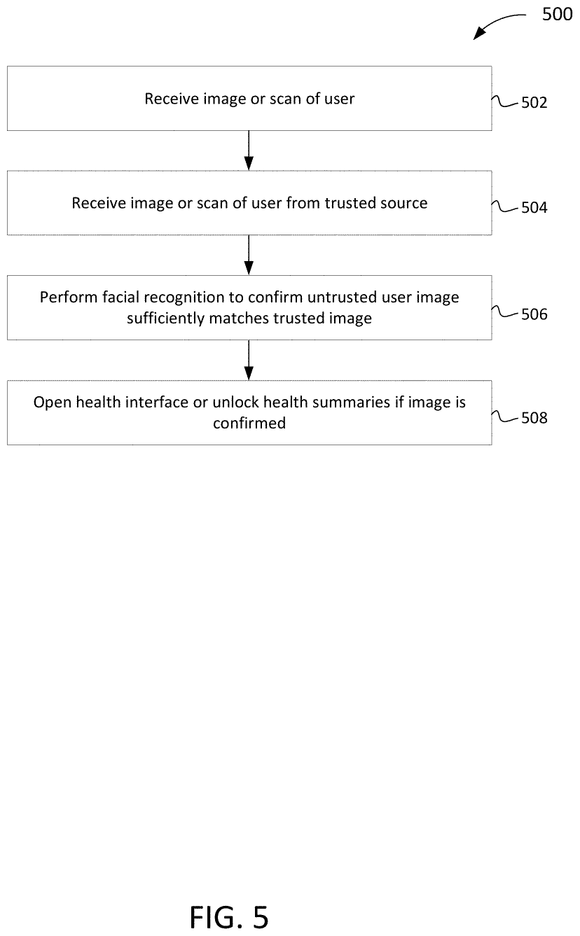

The image assessment module 306 may assess the image from the user (e.g., an untrusted image) to confirm that user's identity. As discussed regarding FIG. 5, the image assessment module 306 may use facial recognition, biomarkers, and/or other information to confirm the image of the user (e.g., using an image from a trusted document).

The information verification module 308 may verify that the document provided by the user is to be trusted. In various embodiments, the information verification module 308 may retrieve information from the document and then confirm the information (e.g., passport identification number) with a trusted data source (such as with the US Government) to verify that the document is authentic.

In various embodiments, the information verification module 308 may receive a scan of a document provided by the user. The information verification module 308 may retrieve information from the information verification module 308 to recognize a type of document (e.g., driver's license, passport, government ID, or the like). The information verification module 308 may retrieve different types of information using, for example, text retrieval or may compare different document templates to the document to determine the type of document. Information verification module 308 may identify one or more data sources to verify the authenticity of the document once the document type is identified. The information verification module 308 may recognize and/or retrieve codes, identification information, identifiers, images, and the like and provide them to the trusted data source and/or retrieve information from the trusted data source to verify the authenticity of the document.

The identification confirmation module 310 may require confirmation of the identity of the user based on the user's untrusted image and an image from the verified, authentic document. In some embodiments, identification confirmation module 310 does not allow the health interface 100 to be accessed or some information to be displayed (e.g., summaries and/or test results), without a confirmation of the identity of the user.

The health information module 312 may be configured to receive and store health information regarding the user. In various embodiments, a health services professional or TIVD system 210 may provide the user smart device 202 with test results, test measurements, test indications, vaccination indications, infection summary, infection information, immunity summary, health information, vaccinations given, applicable health categories (e.g., infected, not infected, immune, and antibody found), and/or the like. The information may be provided by a device associated with the health services professional that provided a test, a health services provider, the TIVD system 210, and/or any other system. The health information module may, in some embodiments, save the information within the health information datastore 316.

In various embodiments, the health information module 312 may retrieve an infection summary or immunity summary from the received information. In some examples, the infection summary or immunity summary may include any or all of the following: when a test was given, and summary of the results of the test such as infected, not infected, immune, not immune, likely infected, likely not infected, likely immune, likely not immune, date when vaccination was given, illnesses tested for, illnesses vaccinate against, and the like) from the received information.

When the health interface 100 is accessed and the user is authorized to receive the information, the health information module 312 may provide the infection summary and/or immunity summary to the user.

In some embodiments, the health information module 312 may perform or receive scoring regarding the potential immunity or infection of the user. The scoring may be based on measurements of testing (e.g., testing of a swab, blood, or the like). The health information module 312 may then determine any number of applicable categories to display as summary information on the health interface 100. The scoring process is described herein.

The image storage datastore 314 is any data storage that may store untrusted images of the user, verified images of the user, scanned documents, retrieved information or images from scanned documents, biomarkers, characteristics of images of the user and/or scanned documents, and/or the like. In various embodiments, the image storage datastore 314 is encrypted.

The health information datastore 316 is any storage that may store any or all health information related to the use. In various embodiments, the health information datastore 316 stores test results, test measurements, test indications, vaccination indications, infection summary, infection information, immunity summary, health information, vaccinations given, applicable health categories (e.g., infected, not infected, immune, and antibody found), scores, thresholds, rules, and/or the like.

A module is any hardware, software, or combination of hardware and software. Although each system depicts one or more modules, it will be appreciated that any module may perform more, or less functionality than what is described herein. Further, each system may have any number of modules, processors, digital devices, or the like.

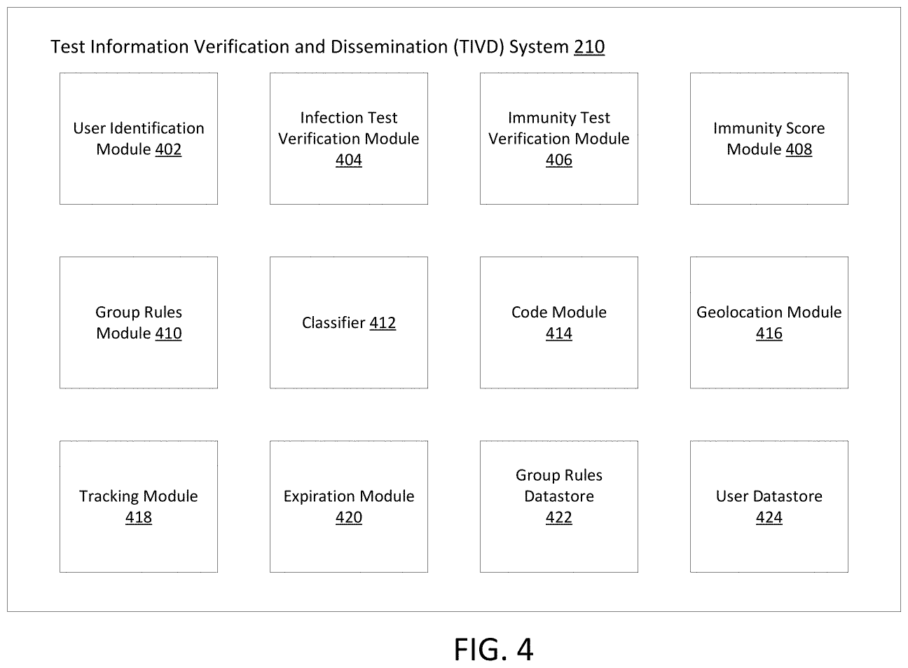

FIG. 4 is a block diagram depicting a TIVD system 210 in some embodiments. The TIVD system 210 may include a user identification module 402, an infection test verification module 404, an immunity test verification module 406, an immunity score module 408, a group rules module 410, a classifier 412, a code module 414, a geolocation module 416, a tracking module 418, an expiration module 420, group rules datastore 422, and user datastore 424.

The user identification module 402 may compare an untrusted image of the user (e.g., received from the user smart device 202) to a trusted image as discussed herein (e.g., through facial recognition, biomarkers, comparison of hash information, characteristic comparisons, and the like).

As discussed herein, the user identification module 402 may receive an untrusted image of the user. The user identification module 402 may receive (e.g., from the user smart device 202) or retrieve (from one or more remote sources) a document containing identifying information or a trusted image. The user identification module 402 may compare the untrusted image (e.g., biomarkers or characteristics of the image) to the trusted image or information (e.g., image recognition) to confirm the identity of the user in the image.

In various embodiments, user identification module 402 may receive a document from the user smart device 202 and confirm the authenticity of the document as described herein.

The user identification module 402 may store the untrusted image, the document, information retrieved from the document, attributes of the untrusted image (e.g., biomarkers or the like), attributes of the trusted image, timestamps of successful facial recognition, and/or timestamps of unsuccessful facial recognition within the user datastore 424.

If the identity of the untrusted image is confirmed to be the user, the identification module 402 may send a confirmed identity indication to the user smart device 202 to indicate that the user's identity is confirmed. In some embodiments, the user identification module 402 may provide an indication that the health interface 100 may be accessed and/or the summary information may be displayed. In various embodiments, the identification module 402 may provide the health interface 100 and/or the health summar(ies) (e.g., infection summary and/or immunity summary) to the user smart device 202 once the identity of the untrusted image is confirmed to be the user.

The user identification module 402 may store the confirmed image of the user (e.g., formerly the untrusted image) within the user datastore 424. In some embodiments, the user identification module 402 may store any documents received from user smart device 202 within a user's record within the user datastore 424, as well as any images retrieved from the documents, and/or information confirming the veracity of the document(s). The user identification module 402 may track each time an untrusted image is received, a document is received, a confirmation process was engaged to confirm the identity of the user, whether the confirmation was successful, what information was provided or not provided based on the comparison, and the destination of the information that was provided.

It may be appreciated that there may be many ways to confirm a user's identity including, but not limited to fingerprint verification with trusted fingerprint information (e.g., by comparison of the current user's fingerprint with a record created by a government agency such as the FBI or police), retina scan, and/or the like.

The infection test verification module 404 may receive and confirm infection test indications and/or infection test information. As discussed herein, the infection test verification module 404 may receive an infection test indication or infection test information from the user smart device 202, a health professional, and/or a remote source (e.g., an HIE, EPIC record, health services provider, and/or the like). The infection test indication may include information indicating that an infection test was made and may identify one or more sources of information that may be contacted to retrieve more information regarding the results of the test.

The infection test verification module 404 may confirm that the information was provided by the particular sender (e.g., by confirming through encryption keys, digital certificates, unique identifiers, white lists, and the like). The infection test verification module 404 may also confirm the authenticity of the infection test indication and/or infection test information through the use of hash tags, encryption keys, digital certificates, unique identifiers, and the like.

In various embodiments, the infection test verification module 404 may retrieve information from any number of remote sources having additional information regarding a particular infection test. For example, the infection test indicator may identify the user and/or related information regarding the laboratory or health service provider that has test results. In some embodiments, the infection indication may indicate a period of time to wait after the test has been given before test results may be available. In some embodiments, the infection test verification module 404 will wait a predetermined period of time (e.g., based on the type of infection test provided) before attempting to retrieve infection test results.

The infection test verification module 404 may retrieve infection test results, infection test information (as discussed herein), and/or other information from the previously identified sources. In some embodiments, the infection test verification module 404 may verify that the sources that are identified as having additional information regarding the test are authorized (e.g., by consulting a list of health service professionals that provide services to the user, a list of authorized laboratories, a list of entities that have relationships with the health service professional that provided the test, and/or the like).

The infection test verification module 404 may assess results from the infection test information (or retrieve the necessary information) to generate and infection summary to provide to the user smart device 202.

The immunity test verification module 406 may receive and confirm immunity test indications, immunity test information, and/or vaccination indications. As discussed herein, the immunity test verification module 406 may receive immunity test indications, immunity test information, and/or vaccination indications from the user smart device 202, a health professional, and/or a remote source (e.g., an HIE, EPIC record, health services provider, and/or the like). The immunity test verification module 406 may confirm that the information was provided by the particular sender (e.g., by confirming through encryption keys, digital certificates, unique identifiers, white lists, and the like). The immunity test verification module 406 may also confirm the authenticity of the immunity test indications, immunity test information, and/or vaccination indications through the use of hash tags, encryption keys, digital certificates, unique identifiers, and the like.

In various embodiments, the immunity test verification module 406 may retrieve remote source identifiers as having additional information regarding a particular immunity test or a vaccination. For example, the immunity test indicator may identify the user and/or related information regarding the laboratory or health service provider that provides the test results. In some embodiments, the immunity test indication may indicate a period of time to wait after the test has been given before test results may be available. In some embodiments, the immunity test verification module 406 will wait a predetermined period of time (e.g., based on the type of infection test provided) before attempting to retrieve infection test results.

The immunity test verification module 406 may retrieve immunity test results, immunity test information (as discussed herein), vaccination information, and/or other information from the previously identified sources. In some embodiments, the immunity test verification module 406 may verify that the sources that are identified as having additional information regarding the test are authorized (e.g., by consulting a list of health service professionals that provide services to the user, a list of authorized laboratories, a list of entities that have relationships with the health service professional that provided the test, and/or the like).

The immunity test verification module 406 may assess results from the immunity test information (or retrieve the necessary information) and/or the vaccination indication to generate and immunity summary to provide to the user smart device 202. In various embodiments, the immunity test verification module 406 may identify test results of immune, not immune, unlikely immune, or likely immune from the results.

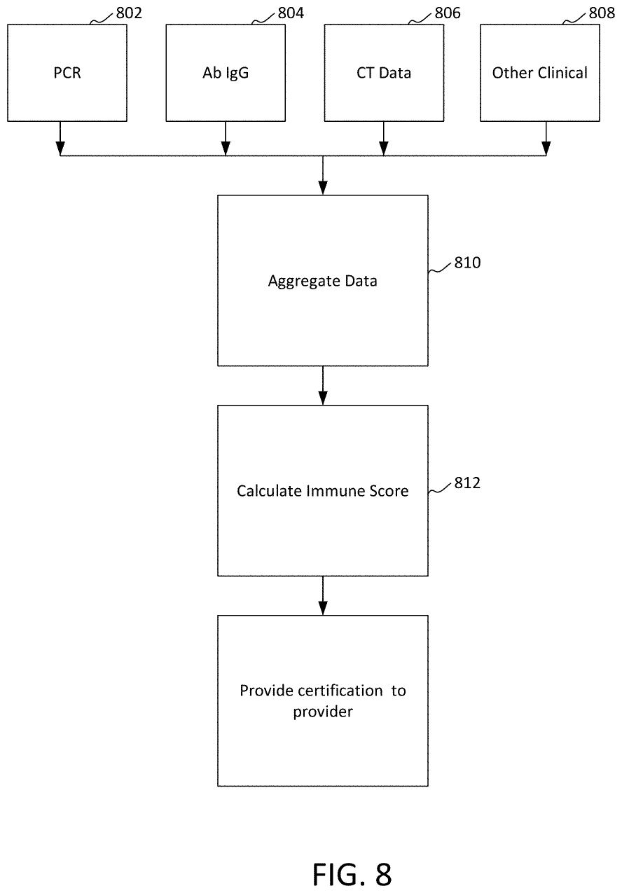

In various embodiments, the immunity test verification module 406 may provide measurements and/or test results associated with an immunity test to the immunity score module 408. The immunity score module 408 may generate an immunity score based on the measurements. It may be appreciated that the immunity score may be calculated in many different ways. An example of an immunity score calculation is discussed regarding FIG. 8.

It will be appreciated that the immunity score module 408 may also calculate an infection score. In some embodiments, the infection test verification module 404 may provide measurements and/or test results associated with an infection test to the immunity score module 408. The immunity score module 408 may generate an infection score based on the measurements. It may be appreciated that the immunity score may be calculated in many different ways

The classifier 412 may determine a category regarding immunity or infection to provide to the user smart device 202 or health interface 100. In one example, the classifier 412 may compare an immunity score to a predetermined category system where different scores represent different immunity categories (e.g., a score at or below a first threshold may indicate “not immune,” a score in between a first and second threshold may indicate “likely not immune,” a score in between a second threshold and a third threshold may indicate “likely immune,” and a fourth threshold may indicate “immune.” The classifier 412 may provide the categories to the user smart device 202 for display on the health interface 100 and/or providing to authorized users.

In another example, the classifier 412 may compare an infection score to a predetermined category system where different scores represent different infection categories (e.g., a score at or below a first threshold may indicate “not infected,” a score in between a first and second threshold may indicate “likely not infected,” a score in between a second threshold and a third threshold may indicate “likely infected,” and a fourth threshold may indicate “infected.” The classifier 412 may provide the categories to the user smart device 202 for display on the health interface 100 and/or providing to authorized users.

The code module 414 may be configured to generate codes to appear in the health interface 100, receive a scanned image of a code (e.g., QR code), and/or receive an identifier from a code. In various embodiments, the health interface 100 displays a QR code or another kind of code that is scannable by digital device. The code module 414 may generate the code that includes a link to send information (e.g., infection test indicators, immunity test indicators, vaccination indicators, health information, and/or the like) to the TIVD system 210 for storage, verification, and/or confirmation. In various embodiments, the code module 414 may generate the code that includes a link to send an indicator to trigger the TIVD system 210 to retrieve information from any number of sources.

When a user registers with the TIVD system 210, the code module 414 may generate the code and incorporating a unique identifier with the user and/or an identifier associated with the user's digital device or TIVD application 300. The code may also include a URL or other identifier that may be utilized to reach out to the TIVD 210. In some embodiments, the code module 414 includes a unique identifier, encryption key, or the like. The unique identifier, encryption key, or the like may be used to confirm communications with the TIVD system 210. For example, a digital device at a venue may scan a user's code from the user's health interface 100. The digital device may retrieve a user identifier, an infection summary, an immunity summary, and a link to the TIVD system 210 from the scanned code. The venue digital device may then send a request regarding the user to the TIVD system 210 to confirm the health information.

The geolocation module 416 may provide safeguards to ensure that an employer, or venue personnel that are checking on the user, are proximate to the user at the time of health information confirmation. In various embodiments, when a venue device reaches out to the TIVD system 210 for health information confirmation, the geolocation module 416 may request or receive geolocation information from both the venue device and the user smart device 202. If the devices are proximate to each other at the time of the request, the geolocation module 416 may enable the confirming process to continue. If, however, the two devices are not proximate to each other at the time of the request, the geolocation module 416 may stop the process because the requester may be attempting to obtain information regarding the user that they are not authorized to receive.

The tracking module 418 may be configured to track information regarding tests and vaccination. If a test is found to be invalid (e.g., the test itself was later discovered to be unreliable, the results were obtained through improper procedure, reagents used in the test were not reliable, the test is expired, or the like), the tracking module 418 may track any number of users who took that test and the TIVD system 210 may update the infection summary and/or the immunity summary accordingly. It will be appreciated that, for example, there are many tests for COVID-19 and some may be found to later be unreliable. By tracking the information of tests and vaccinations (e.g., test identifiers, date of tests, conditions of test, type of test used, and/or the like), the TIVD system 210 may update health information accordingly to ensure the user and the public are protected. In various embodiments, the TIVD system 210 may provide notifications or alerts as needed. The TIVD system 210 may be configured to provide alerts and notifications to the user and other authorized, interested parties (e.g., insurance companies, health providers, and/or employers) regarding the expiration.

The expiration module 420 may track the expiration of any test results and/or vaccinations. Similar to the tracking module 418 discussed herein, the expiration module 420 may track if a vaccination or test has expired and then may update health information (e.g., update the infection summary and/or immunity summary) accordingly. Similarly, the TIVD system 210 may be configured to provide alerts and notifications to the user and other authorized, interested parties (e.g., insurance companies, health providers, and/or employers) regarding the expiration.

The group rules datastore 422 is any data structure or combination of data structures that stores the group rules (e.g., see the discussion regarding group rules module 410). The group rules datastore 422 may be encrypted and require authentication prior to provide group rules as discussed herein.

The user datastore 424 is any data structure or combination of data structures that stores the user health information. Each user may have a record. The record may include identifiers associated with the user or an identifier associated with the user's user smart device 202 (e.g., an identifier or encryption key associated with the user's TIVD application 300). The record may also include health information, infection summaries, immunity summaries, a list of all information provided to the user, a list of all information provided to others (e.g., venue operators, a list of dates and times when information was provided, expiration information, and/or tracking information regarding tests and vaccinations of the user. The group rules datastore 422 may be encrypted and require authentication prior to provide group rules as discussed herein.

In various embodiments, the user datastore 424 may access or be a bockchain that stores the information in a manner, such that any changes are tracked and the information is dependable.

FIG. 5 depicts an example flowchart of confirming a user's identity in some embodiments. It may be appreciated that the user's identity may be confirmed by a user smart device 202, a system on a computer network 214 (e.g., the TIVD system 210), or a combination of both. In various embodiments, the user smart device 202 may (e.g., via the image assessment module 306) utilize facial recognition to confirm the identity of the user. For example, the information capture module 304 may capture information from a government-issued ID as well as an image of the user using a camera on the user smart device 202. Image assessment module 306 may utilize facial recognition to confirm that the image received from the camera on the user smart device 202 is the identity of the user as identified by the government-issued ID.

In step 502, the user smart device 202 may receive an image or scan of the user from a camera or other sensor on the user smart device 202. In various embodiments, the user may take a picture of themselves using the camera. The information capture module 304 may identify the image or scan as an untrusted user image. In some embodiments, the user smart device 202 requires that the user takes an image of themselves within a predetermine time of a request to open the health interface 100 or open the TIVD application 300. For example, the user may request to open the health interface 100. The information capture module 304 may check if an image of the user (e.g., an untrusted image) has been received and confirmed to be trustworthy within a predetermined time (e.g., last twenty minutes, last two minutes, or last minute) before allowing the health interface 100 to open. If an image of the user has not been received and confirmed as trustworthy, then the information capture module 304 may require that user provide a new image from their camera. In other embodiments, the information capture module 304 may require that the user take a new image of themselves ad provide the image to the image assessment module 306 every time the health interface 100 is opened or the TIVD application 300 is opened.

In some embodiments, unless the user has recently had their identity confirmed (e.g., the user may be required to have their identity confirmed before the health interface 100 is displayed), the health interface 100 may not be displayed. In some embodiments, unless the user has recently had their identity confirmed, the health interface 100 may be displayed by the health interface 100 may not display test results and/or immunity results.

In step 504, the information capture module 304 may receive an image or scan of a user from a trusted source such as an identification card. In various embodiments, the trusted identification card is a government ID. It will be appreciated that many different IDs may work including an employer identification card, driver's license, Real ID, Government Issued ID, Green Card, Passport, and/or the like may be utilized.

In some embodiments, the user smart device 202 may retrieve an accepted, trusted user image from another data source on the computer network 214. In one example, the information capture module 304 may provide a request for the trusted image from the TIVD system 210 and/or a third-party entity on the computer network 214.

The TIVD system 210 may store trusted images of any number of users. In some embodiments, the TIVD system 210 may retrieve images of users when the user registers with the TIVD system 210. The TIVD system 210 may retrieve the trusted images from verified databases, government databases, trusted third-party databases, the image storage module 314, and/or the like. Upon request, the TIVD system 210 may provide the image for facial recognition at the user smart device 202. In other embodiments, the TIVD system 210 may receive the recent photograph from the user smart device 202 and may receive the trusted image (e.g., from the user who may have taken a photograph of a trusted identification card or retrieving the trusted image from memory or a third-party source). The TIVD system 210 may subsequently perform facial recognition and provide an indication back to identification confirmation module 310 indicating confirmation of the identity of the user.

In still another embodiment, the user smart device 202 and/or the TIVD system 210 may provide the current image of the user and/or the trusted image to a third-party entity on the computer network 214 for facial recognition. The third-party entity may return an indication of whether the identity of the user was confirmed to the user smart device 202 and/or the TIVD system 210.

In step 506, the image assessment module 306 may perform facial recognition to confirm that the current picture of the user sufficiently matches a trusted image. In various embodiments, the information capture module 304 may identify biomarkers (e.g., points of interest) on the current image and the trusted image for facial recognition. The image assessment module 306 may perform facial recognition based on the biomarkers.

In some embodiments, information capture module 304 may provide the untrusted image and/or the trusted image to the TIVD system 210 for confirmation. The TIVD system 210 may perform facial recognition or provide other biomarker comparisons to confirm the user. The TIVD system 210 may provide the confirmation back to the information capture module 304.

In various embodiments, the information capture module 304 may receive a trusted image (e.g., from the user's driver's license) and the image assessment module 306 may identify biomarkers of the trusted image. The biomarkers (or a hash) may be stored in the image storage module 314. When the user wishes to have their identity verified, the user may provide a current image taken with a camera on the user smart device 202 and the image assessment module 306 (or the identification confirmation module 310) may compare biomarkers of the new, current image against the previously stored biomarkers (e.g., using the hash) stored in the image storage module 314.

Similarly, the biomarkers (or the hash) may be stored at the TIVD system 210 and retrieved as needed for confirmation of the user's identity. In some embodiments, the biomarkers stored on a source on the computer network 214 (e.g., at the TIVD system 210 or another source) may be anonymized or identified a value or hash that is not personally identifiable. The image assessment module 306 may provide a software identifier or image identifier associated with an application, the user smart device 202, or the like and the TIVD system 210 may retrieve biomarkers or a hash of biomarkers from storage for comparison with the biomarkers of the current photograph.

In step 508, if the image assessment module 306 or the identification confirmation module 310 confirms the identity of the user from the user's new current photograph (e.g., or the TIVD system 210 confirms the user's identity), the image assessment module 306 or the identification confirmation module 310 may unlock access to the health interface 100 or unlock health summaries (e.g., test results/immunity results) for display on the health interface 100.

In some embodiments, if the image assessment module 306 or the identification confirmation module 310 confirms the identity of the user from the user's new current photograph (e.g., or the TIVD system 210 confirms the user's identity), the image assessment module 306 may enable the health interface 100 to indicate that the user's identify has been confirmed. If the image assessment module 306 does not confirm the identity of the user from the user's new current photograph, the health interface 100 may indicate that the users identify has not been confirmed. In this example, if the user's identity has not been confirmed, the health interface 100 may indicate that the health summary information (e.g., infection summary and health summary) are not available or are only available pending confirmation of user's identity.

If the TIVD system 210 performs the confirmation of the user's identity, the user identification module 402 may receive the user's current photograph (e.g., image or scan) from the user smart device 202. As discussed herein, the user identification module 402 may receive a trusted image or a scan of a document with a trusted image. The user identification module 402 may retrieve the trusted image from the document. In some embodiments, the user identification module 402 and/or the image assessment module 306 may confirm the validity of the document by comparing the document to known characteristics of trusted documents (e.g., a US passport may have expected information in a particular format on the page containing an image), confirming identifiers (e.g., confirming name and/or ID numbers associated with the user's passport with a US government agency that may track information and/or issues passports), confirm against other known or previously collected trusted documents (e.g., a user's previously scanned driver's license), and/or the like.

An infection test is a test to determine if a particular person is infected with a particular virus or illness. An antibody test is a test to determine if a particular person has antibodies or other indicators of immunity.

An infection summary includes infection information that may indicate if a user is infected or not infected. The infection summary may also include a date or other information regarding when the last infection test was given to the user.

An immunity summary may include information that may indicate if a user has been vaccinated (i.e., and therefore assumed to be immune) or has been tested for antibodies or other characteristics of immunity. The immunity summary may include an indication if the user is immune or not immune. In some embodiments, the immunity summary includes a date of vaccination or a date of the last immunity test.

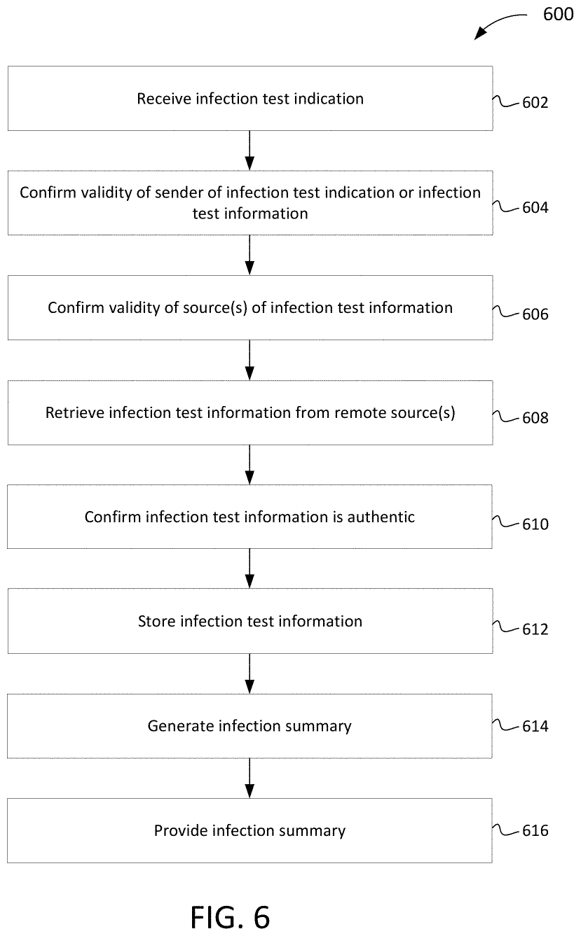

FIG. 6 is a flowchart for receiving and verifying infection test information in some embodiments. In some embodiments, the TIVD system 210 may receive an indication that an infection test has been performed. The TIVD system 210 may subsequently retrieve information regarding the infection test and/or summarize the test results. In the process, the TIVD system 210 may confirm the authenticity of the source of the information and/or authenticity of the information itself. Subsequently, the TIVD system 210 may provide information (e.g., to the health interface 100) as an infection summary.

While the discussion regarding FIG. 6 is directed to infection tests and infection test results, it will be appreciated that the process may be similar for immunity tests (e.g., vaccinations and/or antibody tests), and immunity test results. In various embodiments, the TIVD system 210 may receive virus-related test results and/or receive a virus-related test indication. A virus-related test may be an infection test or an immunity test. Similarly, virus-related test results are results that are related to an infection test or an immunity test. In this example, the term “virus-related” is a broader term that covers both “infection” and “immunity.”

It will be appreciated that the TIVD system 210 may receive any number of infection test indications regarding any number of people (e.g., across a city, state, country, continent, or the like). The infection test indications may be provided from any number of different health professional entities and associated digital devices. A health professional may utilize an associated digital device or a user's smart device to upload or otherwise provide the infection test indication (e.g., after the health professional conducted an infection test on a user). It will be appreciated that a testing center may utilize one or more digital devices to upload infection test indications to the TIVD system 210 and/or a related health service organization, EPIC system, HIE system, laboratory, and/or the like. In various embodiments, the TIVD system 210 may retrieve different infection test information for different people from a wide variety of different, unrelated laboratories, EPIC systems, health services organizations, health services providers, HIE systems, and the like.

The TIVD system 210 may retrieve infection health information (e.g., the results of the infection test and when the infection test was given. The TIVD system 210 may also confirm the source of the infection health information to confirm validity.

In some embodiments, infection test information, including test results and date of the test is provided directly to the user smart device 202 without the user smart device 202 retrieving information and/or receiving an infection test indication. The infection test information may be provided directly from a health provider giving the test or a remote source that pushes only needed health data to the TIVD system 210 (e.g., using a secure connection and upon agreements between the parties).

After confirming validity, the TIVD system 210 may provide an infection summary to the tested person's the user smart device 202 for display on the health interface 100. In some embodiments, the TIVD system 210 may provide all or parts of the infection summary (and/or related data) to other entities (e.g., an employer, venue operator, or the like).

While many of the examples discussed herein are directed to testing for COVID infections, in some embodiments, the TIVD system 210 may receive and/or confirm the validity of infection test information of a wide variety of different illnesses. It may be appreciated that the TIVD system 210 may be utilized to confirm and provide infection test results regarding COVID for one person while confirming and providing infection test results regarding an unrelated influenza virus for a second person.

In step 602, the immunity test verification module 406 receives an infection test indication from either the user smart device 202 or another device. In various embodiments, a user may be tested for infection. The health professional may input the infection test indication to the user smart device 202 (using an interactive component of the health interface 100) or input the infection test indication into their own digital device. The health professional's digital device may, in one example, scan the code of the user's the health interface 100 to access an interface (e.g., a web page interface, application interface, or API) to upload the infection test indication and/or infection test information (e.g., date infection test was given, name of the test) as well as a user identifier (e.g., the user identifier may be taken by the health professional's digital device from the code).

In another example, the health professional's digital device may upload the infection test indication and/or infection test information (e.g., date infection test was given, name of test, and/or results of the test) to a health service provider to store in health records. In one example, the health service provider may store the infection test indication and/or infection test information in a record of an EPIC system, HIE system, health system, facility system, or the like. The EPIC system, HIE system, health system, facility system, or the like may, in some embodiments, provide the infection test indication and/or infection test information to the user smart device 202.

An infection test indication may include an identifier indicating where information related to the infection test may be retrieved (e.g., a database or record stored or managed by an EPIC system, HIE system, health system, facility system, or the like). In some embodiments, the immunity test verification module 406 may retrieve all or some infection test information. An infection test indication may include an indication that an infection test was performed, an infection test identifier (e.g., a unique identifier associated with the infection test), and metadata associated with the infection test. The metadata may indicate a source where test results may be retrieved (e.g., a health provider, clinic, laboratory, EPIC record, or the like where test results may be retrieved.

In some embodiments, instead of an infection test indicator, the user smart device 202 or another device may provide infection test information. For example, infection test information may be uploaded to the TIVD system 210 using the user smart device 202 or a device associated with testing the user. In some embodiments, the user and/or the health professional providing the test may upload all or some of the infection test information. Infection test information may include, for example, date of infection test, type of infection test (e.g., blood or swab), infection test identifiers (e.g., specific identification of the test such as the test name or code), infection test metadata (e.g., how delivered, reagents used, conditions of the test, location of the test), condition of user (e.g., displaying symptoms, not displaying symptoms, distressed, immediate and short term reactions to the test), laboratory or health facility that are to provide infection test results (if any), test results (if available), and/or the like.

It will be appreciated that any or all of the infection test information may be provided to the TIVD system 210 as well as other devices in communication over the computer network 214. For example, all or some of the infection test information may be stored locally by the health professional providing the test, in any number of health records (e.g., physical and electronic), health facilities (e.g., hospitals and clinics), laboratories, and/or health providers.

In step 604, the immunity test verification module 406 may optionally confirm the validity of the sender of the infection test indication or the infection test information. For example, the TIVD system 210 may utilize encryption keys and/or other secure identifiers to confirm that the device providing the infection test indication is authentic and/or authorized. In one example, the TIVD system 210 may utilize encryption keys and/or other secure identifiers to confirm that the sender (e.g., the user smart device 202, a digital device associated with the health services professional, or any device providing the infection test information and/or infection test indication) is authentic and/or are authorized. For example, the TIVD system 210 may require that the infection test indication must be encrypted by a health service's encryption key and/or may confirm a unique identifier (e.g., MAC address, digital signature, certificate, and/or the like) to confirm the sender. In another example, the TIVD system 210 may require that the infection test indication must be encrypted by a user's encryption key. In this example, a user may upload the infection test indication to the TIVD system 210 using an application's encryption key. In another example, a health services professional giving the test may scan the user's code from the health interface 100 and the user's code may include the user's encryption key to assist in encrypting the infection test information.

In step 606, the immunity test verification module 406 may confirm the validity of any number of remote sources prior to retrieving information. For example, the TIVD system 210 may receive an indication that a particular remote source (e.g., EPIC system) controls access to a user's health record containing infection test information. The immunity test verification module 406 may confirm that the particular remote source is a valid, known system of health records (e.g., on a trusted white list that is checked by the TIVD system 210). In some embodiments, the immunity test verification module 406 may check of the particular remote source is associated with the user's known health service provider (e.g., the user is a member of Kaiser's health system and the infection test indication indicates that the infection test information is accessible through a third-party health care system in another country). If there is an inconsistency or the source is not sufficiently trustworthy, the TIVD system 210 may provide an indication to the user (e.g., via the health interface 100) or other authorized user that a confirmed infection test summary is not available.

In step 608, the immunity test verification module 406 may retrieve infection test results information from any number of remote sources. For example, the user or health professional administering the infection test may provide an infection test identifier and/or metadata that indicates that an infection test was given as well as an indication of where additional information may be received (e.g., which record of an EPIC system, HIE system, health system, facility system, or the like).

In some embodiments, the immunity test verification module 406 receives all of the necessary infection test information from a single source. The infection test information may be from a health record, from a digital device of a health service provider, from a digital device of the health professional providing the infection test, from a health information exchange (HIE), EPIC system, or the like.

It will be appreciated that the TIVD system 210 may be required to have an account, encryption keys, and sufficient security to communicate with a digital device of a health service provider, the digital device of the health professional providing the infection test, the health information exchange (HIE), EPIC system and/or the like. Any or all sources may require the TIVD system 210 to provide encryption keys, digital signatures, certificates, codes, passwords, and the like to communicate with the sources. Further, any or all sources may require independent confirmation of the user that is associated with any of the infection test information.

In some embodiments, the TIVD system 210 may receive information from an electronic HIE. The TIVD system 210 may be a member of the HIE. In one example, the TIVD system 210 may establish a secure, encrypted connection with the electronic HIE and provide query or otherwise access an electronic record associated with the user that was tested (e.g., using a user identifier from or based on the infection test indication) or other information. In some embodiments, the TIVD system 210 may identify and retrieve information only related to the infection test by using the infection test indication and/or infection test information. It will be appreciated that the TIVD system 210 may only retrieve information of interest to confirm and verify the infection test. In some embodiments, the TIVD system 210 may retrieve information from the record(s) of the HIE, including a confirmation when the infection test was completed, the type of infection test used, infection test names/identifiers, results of the test, and/or the like. The TIVD system 210 may store any or all of the information.

In some embodiments, the TIVD system 210 may receive infection test results (i.e., results such as measurements or categories of health condition) from a health services provider, tester, or the like. If the information is assessed and confirmed as being from a trusted source and the information is checked for trustworthiness, the TIVD system 210 may store the information and provide an infection summary based on the information. In some embodiments, the TIVD system 210 may retrieve confirming information from one or more other, known, trusted sources to confirm the immunity test results prior to providing an immunity summary (i.e., the TIVD system 210 may conclude that trusted information is not yet received and so cannot provide an infection summary. In another example, the TIVD system 210 may provide all or some of the infection test results to one or more other, known, trusted sources with a request that the trusted source (e.g., health services professional, HIE, or the like) provide an indication that the information is accurate or inaccurate.

In step 610, the immunity test verification module 406 may confirm that some or all of the infection test information is authentic. For example, if the infection test information is retrieved from a known, secure source (such as an EPIC record maintained by a trusted system and or HIE), the immunity test verification module 406 may assume authenticity. In some embodiments, the immunity test verification module 406 may provide challenges, request encryption keys, decrypt information using encryption keys, utilize digital certificates, and/or the like to confirm that the source is authentic. In some embodiments, the immunity test verification module 406 may confirm that the infection test information received from the source is authentic by scanning the information for malware, confirming the information using a known hash received from another network or another source, decrypting the information with a trusted key, and/or the like. In these examples, the immunity test verification module 406 may separately confirm the source of information as being authentic and all or some of the infection test information as being authentic prior to storing or providing the information.

Alternately, in some embodiments, the TIVD system 210 may request any or all infection test information using a user identifier that this not associated with the user's personal information. The source of information may confirm the requested information is associated with the identifier and provide the requested information without providing any additional or personal information regarding the user.

In step 612, the immunity test verification module 406 may store any or all of the infection test information for that particular user in the user datastore 424. It will be appreciated that, in some embodiments, the TIVD system 210 may store only a subset of information such as test results and user identifiers. A user identifier may be an identifier associated with a user, a user device 202, a certificate, an encryption key, or the like. In some embodiments, the user identifier may not be personally identifiable.

In step 614, the group rules module 410 may generate an infection summary. The infection summary may include, in some embodiments, test results, date of test, and type of test (e.g., name of test, category of test such as oral, swab, blood sample, reagent used, and/or the like). In some embodiments, the infection test information may include measurements based on infection test and/or an indication of likelihood of infection. In various embodiments, the group rules module 410 may compare one or more predetermined threshold(s) to the measurement(s) or likelihood of infection to determine a category of infection (e.g., infected, likely infected, likely not infected, or and infected). The infection summary may include the category of infection determined by the group rules module 410 or a category of infection provided by one or more sources. The infection summary may also include the most recent infection test. In some embodiments, the infection summary may include a user identifier associated with the user. The user identifier may be a name of the user that was tested. In some embodiments, the infection summary and the user identifier may be provided to an authorized third-party. The authorized third-party may be authorized by the user and/or the health service that provides service to the user.

In step 616, the group rules module 410 provides the infection summary to the user to appear on the health interface 100.

In some embodiments, a user's health information (e.g., regarding testing or vaccination) may be stored in an anonymized form. In one example, the health information stored in the user datastore 424 may be associated with an identifier that is linked to a user's user smart device 202 and/or the user's health interface 100. The stored health information, however, may have all personalized information removed prior to storage. Similarly, while test summaries, vaccination summaries, and/or other information may be provided to the user smart device 202 and/or other authorized device, the information may not (in some embodiments) include the user's name or other personally identifying information.

Further, in some embodiments, a unique identifier may be used to refer to the user, the user's account, the user's TIVD application, and/or the like. The unique identifier may be utilized by remote sources and/or other devices to store information and/or provide information without sending any personally identifying information. For example, an EPIC record may include the unique identifier used by the TIVD system 210. The TIVD system 210 may request test information regarding the unique identifier, and the EPIC system may retrieve and provide health information using the unique identifier. The health information provided by the EPIC system may not (in this example) include any personally identifying information.

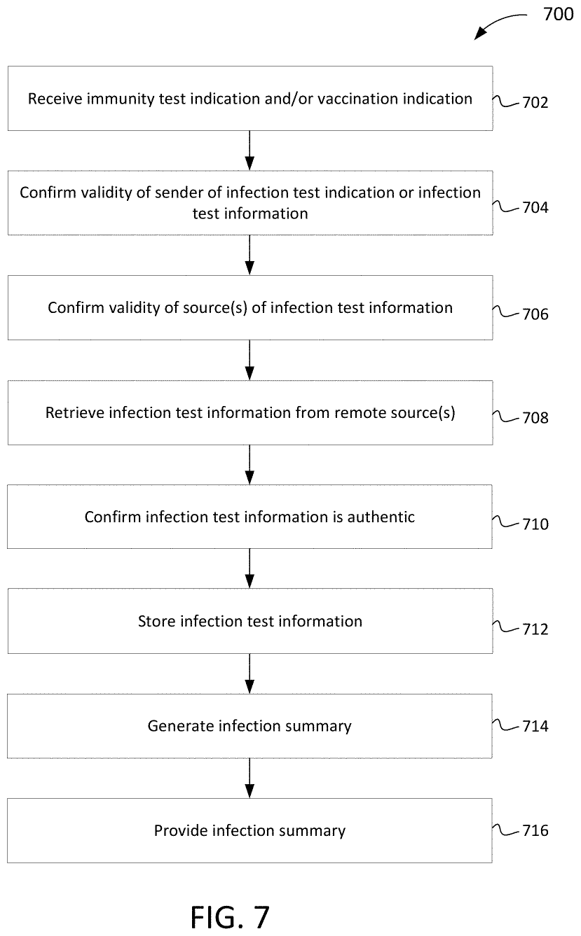

FIG. 7 is a flowchart for receiving and verifying immunity information in some embodiments. In some embodiments, the TIVD system 210 may receive an indication that an immunity test (e.g., a test for antibodies) has been performed. The TIVD system 210 may subsequently retrieve information regarding the immunity test and/or summarize the test results. Further, the TIVD system 210 may receive an indication that a vaccination has been performed. In the process, the TIVD system 210 may confirm authenticity of the source of the information and/or authenticity of the information itself. Subsequently, the TIVD system 210 may provide information (e.g., to the health interface 100) as an immunity summary.

It will be appreciated that the TIVD system 210 may receive any number of immunity test indications and/or vaccination indications (e.g., an indication that the user was vaccinated) regarding any number of people (e.g., across a city, state, country, continent, or the like). The immunity test indications and/or the vaccination indication may be provided from any number of different health professional entities and associated digital devices. A health professional may utilize an associated digital device or a user's smart device to upload or otherwise provide the immunity test indication (e.g., after the health professional conducted an antibody test on a user) or vaccination.

It will be appreciated that a testing center may utilize one or more digital devices to upload immunity test indications and/or vaccination indications to the TIVD system 210 and/or a related health service organization, EPIC system, HIE system, laboratory and/or the like. In various embodiments, the TIVD system 210 may retrieve different immunity test indications and/or vaccination indications for different people from a wide variety of different, unrelated laboratories, EPIC systems, health services organizations, health services providers, HIE systems and the like.

The TIVD system 210 may retrieve immunity health information (e.g., the results of the immunity test, when the immunity test was given, when the vaccination was given, type of immunity test, type of vaccine, name of immunity test, name of the vaccine, the particular virus or illness that was vaccinated against, the date of vaccination, expected duration of effectiveness, and the like). The TIVD system 210 may also confirm the source of the immunity health information to confirm validity.

In some embodiments, immunity health information, including test results, date of immunity test, and/or date of the vaccination is provided directly to the user smart device 202 without the user smart device 202 retrieving information and/or receiving an immunity test indication. The immunity health information may be provided directly from a health provider giving the test or a remote source that pushes only needed health data to the TIVD system 210 (e.g., using a secure connection and upon agreements between the parties).

After confirming validity, the TIVD system 210 may provide an immunity summary to the person's the user smart device 202 for display on the health interface 100. In some embodiments, the TIVD system 210 may provide all or parts of the immunity summary (and/or related data) to other entities (e.g., an employer, venue operator, or the like).

While many of the example discussed herein are directed to immunity for COVID infections, in some embodiments, the TIVD system 210 may receive and/or confirm validity of immunity information of a wide variety of different illnesses (e.g., vaccinations for two or more different illnesses, antibody testing for two or more different illnesses, and/or any combination). It may be appreciated that the TIVD system 210 may be utilized to confirm and provide immunity test results regarding COVID for one person while confirming and providing immunity test results regarding an unrelated influenza virus for a second person.

In step 702, the immunity test verification module 406 receives an immunity test indication from either the user smart device 202 or another device. In various embodiments, a user may be tested for immunity. The health professional may input the immunity test indication to the user smart device 202 (using an interactive component of the health interface 100) or input the immunity test indication into their own digital device. The health professional's digital device may, in one example, scan the code of the user's health interface 100 to access an interface (e.g., a web page interface, application interface, or API) to upload the immunity test indication and/or immunity information (e.g., date infection test was given, name of test) as well as a user identifier (e.g., the user identifier may be taken by the health professional's digital device from the code).

Similarly, the immunity score module 408 may receive a vaccination indication from either the user smart device 202 or another device. In various embodiments, a user may receive a vaccination. The health professional may input the vaccination indication to the user smart device 202 (using an interactive component of the health interface 100) or input the immunity indication into their own digital device. The health professional's digital device may, in one example, scan the code of the user's the health interface 100 to access an interface (e.g., a web page interface, application interface, or API) to upload the vaccination indication and/or immunity information (e.g., date vaccination was given, name of vaccination) as well as a user identifier (e.g., the user identifier may be taken by the health professional's digital device from the code).

In another example, the health professional's digital device may upload the immunity test indication, immunity health information (e.g., date infection test was given, name of test, and/or results of test), and/or vaccination to a health service provider to store in health records. In one example, the health service provider may store the immunity test indication, immunity health information, and/or vaccination in a record of an EPIC system, HIE system, health system, facility system, or the like. The EPIC system, HIE system, health system, facility system, or the like may, in some embodiments, provide the infection test indication and/or infection test information to the user smart device 202.

An immunity test indication may include an identifier indicating where information related to the immunity test may be retrieved (e.g., a database or record stored or managed by an EPIC system, HIE system, health system, facility system, or the like). Similarly, a vaccination indication may include an identifier indicating where information related to the vaccination may be retrieved. In some embodiments, the immunity test verification module 406 and/or the immunity score module 408 may retrieve all or some immunity information. An immunity indication may include an indication that an immunity test was performed, vaccination was performed, an immunity test identifier (e.g., a unique identifier associated with the immunity test), a vaccination test identifier, and/or metadata associated with the immunity test and/or vaccination. The metadata may indicate a source where test results may be retrieved (e.g., a health provider, clinic, laboratory, EPIC record, or the like where test results may be retrieved).

In some embodiments, instead of an immunity indicator, the user smart device 202 or another device may provide immunity health information. For example, immunity health information may be uploaded to the TIVD system 210 using the user smart device 202 or a device associated with testing or vaccination of the user. In some embodiments, the user and/or the health professional providing the test or vaccination may upload all or some of the immunity health information. Immunity health information may include, for example, date of infection test, type of infection test (e.g., blood or swab), infection test identifiers (e.g., specific identification of the test such as the test name or code), infection test metadata (e.g., how delivered, reagents used, conditions of the test, location of the test), condition of user (e.g., displaying symptoms, not displaying symptoms, distressed, immediate and short term reactions to the test), laboratory or health facility that are to provide infection test results (if any), test results (if available), and/or the like.

It will be appreciated that any or all of the immunity health information may be provided to the TIVD system 210 as well as other devices in communication over the computer network 214. For example, all or some of the immunity health information may be stored locally by the health professional providing the test, in any number of health records (e.g., physical and electronic), health facilities (e.g., hospitals and clinics), laboratories, and/or health providers.

In step 704, the immunity test verification module 406 and/or the immunity score module 408 may optionally confirm the validity of the sender of the immunity test indication, immunity information, or vaccination information. For example, the TIVD system 210 may utilize encryption keys and/or other secure identifiers to confirm that the device providing the immunity test indication, immunity information, or vaccination information is authentic and/or authorized. In one example, the TIVD system 210 may utilize encryption keys and/or other secure identifiers to confirm that the sender (e.g., the user smart device 202, a digital device associated with the health services professional, or any device providing the immunity test indication, immunity information, or vaccination information) is authentic and/or are authorized. For example, the TIVD system 210 may require that the immunity test indication, immunity information, or vaccination information must be encrypted by a health service's encryption key and/or may confirm a unique identifier (e.g., MAC address, digital signature, certificate, and/or the like) to confirm the sender. In another example, the TIVD system 210 may require that the immunity test indication, immunity information, or vaccination information must be encrypted by a user's encryption key. In this example, a user may upload the immunity test indication or vaccination indication to the TIVD system 210 using an application's encryption key. In another example, a health services professional giving the test may scan the user's code from the health interface 100 and the user's code may include the user's encryption key to assist in encrypting the immunity test indication, immunity information, or vaccination information.

In step 706, the immunity test verification module 406 and/or the immunity score module 408 may confirm the validity of any number of remote sources prior to retrieving information. For example, the TIVD system 210 may receive an indication that the particular remote source (e.g., EPIC system) controls access to a user's health record containing infection test information. the immunity test verification module 406 may confirm that the particular remote source is a valid, known system of health records (e.g., on a trusted white list that is checked by the TIVD system 210). In some embodiments, the immunity test verification module 406 and/or the immunity score module 408 may check of the particular remote source is associated with the user's known health service provider (e.g., the user is a member of Kaiser's health system and the infection test indication indicates that the infection test information is accessible through a third-party health care system in another country). If there is an inconsistency or the source is not sufficiently trustworthy, the TIVD system 210 may provide an indication to the user (e.g., via the health interface 100) or other authorized user that a confirmed immunity summary is not available.

In step 708, the immunity test verification module 406 and/or the immunity score module 408 may retrieve immunity health information from any number of remote sources. For example, the user or health professional administering the immunity test may provide an immunity test identifier and/or metadata that indicates that an infection test was given as well as an indication of where additional information may be received (e.g., which record of an EPIC system, HIE system, health system, facility system, or the like). Similarly, the user or health professional administering a vaccination may provide a vaccination identifier and/or metadata that indicates that an vaccination was given as well as an indication of where additional information may be received (e.g., which record of an EPIC system, HIE system, health system, facility system, or the like).

In some embodiments, the immunity test verification module 406 and/or the immunity score module 408 receives all of the necessary immunity health information from a single source. The immunity health information may be from a health record, from a digital device of a health service provider, from a digital device of the health professional providing the infection test, from a health information exchange (HIE), EPIC system, or the like.

It will be appreciated that the TIVD system 210 may be required to have an account, encryption keys, and sufficient security to communicate with a digital device of a health service provider, the digital device of the health professional providing the immunity test or vaccination, the health information exchange (HIE), EPIC system and/or the like. Any or all sources may require the TIVD system 210 to provide encryption keys, digital signatures, certificates, codes, passwords, and the like to communicate with the sources. Further, any or all sources may require independent confirmation of the user that is associated with any of the immunity health information.

In some embodiments, the TIVD system 210 may receive information from an electronic HIE. The TIVD system 210 may be a member of the HIE. In one example, the TIVD system 210 may establish a secure, encrypted connection with the electronic HIE and provide query or otherwise access an electronic record associated with the user that was tested (e.g., using a user identifier from or based on the immunity test indication) or vaccinated. In some embodiments, the TIVD system 210 may identify and retrieve information only related to the infection test by using the infection test indication and/or infection test information. It will be appreciated that the TIVD system 210 may only retrieve information of interest to confirm and verify the immunity test. In some embodiments, the TIVD system 210 may retrieve information from the record(s) of the HIE, including a confirmation when the immunity test was completed, the type of immunity test used, infection test names/identifiers, results of the test, and/or the like. The TIVD system 210 may store any or all of the information.

In some embodiments, the TIVD system 210 may receive immunity test results (i.e., results such as measurements or categories of health condition) from a health services provider, tester, or the like. If the information is assessed and confirmed as being from a trusted source and the information is checked for trustworthiness, the TIVD system 210 may store the information and provide an immunity summary based on the information. In some embodiments, the TIVD system 210 may retrieve confirming information from one or more other, known, trusted sources to confirm the immunity test results prior to providing an immunity summary (i.e., the TIVD system 210 may conclude that trusted information is not yet received and so cannot provide an immunity summary. In another example, the TIVD system 210 may provide all or some of the immunity test results to one or more other, known, trusted sources with a request that the trusted source (e.g., health services professional, HIE, or the like) provide an indication that the information is accurate or inaccurate.

In step 710, the immunity test verification module 406 and/or the immunity score module 408 may confirm that some or all of the immunity health information is authentic. For example, if the immunity health information is retrieved from a known, secure source (such as an EPIC record maintained by a trusted system and or HIE), the/immunity test verification module 406 and/or the immunity score module 408 may assume authenticity. In some embodiments, the immunity test verification module 406 and/or the immunity score module 408 may provide challenges, request encryption keys, decrypt information using encryption keys, utilize digital certificates, and/or the like to confirm that the source is authentic. In some embodiments, the immunity test verification module 406 and/or the immunity score module 408 may confirm that the immunity health information received from the source is authentic by scanning the information for malware, confirming the information using a known hash received from another network or another source, decrypting the information with a trusted key, and/or the like. In these examples, the immunity test verification module 406 may separately confirm the source of the information as being authentic and all or some of the immunity health information as being authentic prior to storing or providing the information.

Alternately, in some embodiments, the TIVD system 210 may request any or all immunity health information using a user identifier that this not associated with the user's personal information. The source of the information may confirm the requested information is associated with the identifier and provide the requested information without providing any additional or personal information regarding the user.

In step 712, the immunity test verification module 406 and/or the immunity score module 408 may store any or all of the immunity health information for that particular user in the user datastore 424. It will be appreciated that, in some embodiments, the TIVD system 210 may store only a subset of information such as test results and user identifiers. A user identifier may be an identifier associated with a user, a user device 202, a certificate, an encryption key, or the like. In some embodiments, the user identifier may not be personally identifiable.

In step 714, the group rules module 410 may generate an immunity summary. The immunity summary may include, in some embodiments, test results, date of test, and type of test (e.g., name of test, category of test such as oral, swab, blood sample, reagent used, and/or the like). In some embodiments, the immunity may include measurements based on infection test and/or an indication of likelihood of infection. In various embodiments, the immunity test verification module 406 may compare one or more predetermined threshold(s) to the measurement(s) or likelihood of immunity to determine a category of immunity (e.g., immune, likely immune, likely not immune, or and immune). The immune summary may include the category of immunity determined by the immunity test verification module 406 or a category of immunity provided by one or more sources. The immunity summary may also include the most recent immunity test. The immunity summary may also include an indication of immune if the user is provided a vaccination. The immunity summary may include the type of vaccination given, the particular virus or illness that was vaccinated against, the date of vaccination, expected duration of effectiveness, and/or the like.

In some embodiments, the immunity summary may include a user identifier associated with the user. The user identifier may be a name of the user that was tested. In some embodiments, the infection summary and the user identifier may be provided to an authorized third-party. The authorized third-party may be authorized by the user and/or the health service that provides service to the user.

In step 716, the group rules module 410 provides the infection summary to the user to appear on the health interface 100. In various embodiments, the group rules module 410 may provide the infection summary to any authorized requester (e.g., authorized by the user and/or their health provider).

FIG. 8 is a flow diagram of calculating a score based on test measurements in some embodiments. As discussed herein, the infection test verification module 404 and/or the immunity test verification module 406 may receive health information and/or test results related to different tests. Measurements from the test results may be scored to determine a classification of infection (e.g., an infection classification) or a classification of immunity (e.g., an immunity classification). As discussed herein, the infection classification may indicate if the user is infected or not infected. Further, in some embodiments, the infection classification may indicate if the user is “likely infected” or “likely not infected” or other variations. Similarly, as discussed herein, the immunity classification may indicate if the user is immune or not immune to a particular illness or virus. Further, in some embodiments, the immunity classification may indicate if the user is “likely immune” or “likely not immune” or other variations. As also discussed herein, once a score is calculated, the score may be compared to different thresholds to determine the classification.

In some embodiments, different thresholds may be applied depending on the situation or context. In one example, a standard set of predetermined thresholds may be applied when the categories will be provided to different user's the user smart device 202. Different group rules, however, may include any number of sets of different thresholds. For example, a group rule associated with a hospital employer may have different thresholds than a group rule associated with a car manufacturer.

In various embodiments, venues and employers may have any number of group rules. In one example, a large employer with offices in many different counties and/or states may provide different rules depending on location of the user device (e.g., the user device in New York City may require different, more stringent thresholds than a user device in a small town in Jewell, Okla.), office location, job (e.g., the user interacts with many people or few people), individual (e.g., the user may be older or may have conditions that put them “at risk”), or the like. As the user changes locations, jobs, or the like, the TIVD system 210 may receive that information and provide the thresholds based on the applicable group rules based on those changes.

In the example of FIG. 8, scoring of immunity test measurements is discussed. It will be appreciated that similar or different methodologies may be utilized to score infections or other conditions. In this example, the TIVD system 210 may retrieve immunity test measurements from any number of sources.

The immunity score module 408 may retrieve relevant test results from the immunity test measurements. In one example, the TIVD system 210 may retrieve immunity test information that includes test measurements such as polymerise chain reaction (PCR) test measurements 802, immunoglobulin G (Ab IgG) test measurements 804, and CT Data 806. The test measurements may also include other clinical test information 808.

The immunity score module 408 may aggregate data in step 810 and calculate an immunity score 812 based on the aggregated data.

Subsequently, the classifier 412 may compare the score to classifications (e.g., related to group rules or default classifications) to provide the results (e.g., results that are certified based on the security, retrieval from trusted sources, and other steps discussed herein).

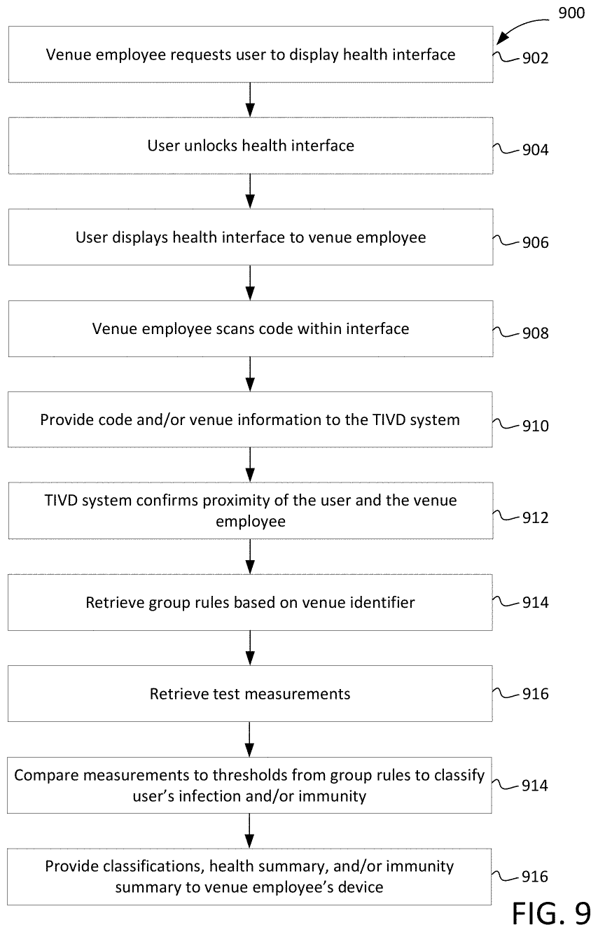

FIG. 9 is an example method of providing an infection summary and/or an immunity summary to a venue employee in some embodiments. As discussed herein, different venues may have different classifications based on different sets of thresholds. A hospital may require a greater degree of certainty regarding immunity or an uninfected status than a job where only a few employees may be present in the facility and the employees may be able to be socially distanced.

In some embodiments, a venue employee (e.g., attendant, host, security, or the like) may have an application (e.g., a TIVD application) on a digital device. One or more venues may register with the TIVD system 210 and provide a criteria for confirming the user's health status. The criteria may include a set of thresholds, a methodology for scoring, requirements for security (e.g., encryption keys and digital certificates) for authenticating the venue employee's devices and/or the user smart device 202.

In some embodiments, a venue may rely on the summaries and/or classifications provided by the user's health interface 100. Other venues may require further confirmation by scanning the code on the health interface 100 and/or requiring different conditions be met (as described herein).

In step 902, a venue employee may request that the user present the health interface 100 to a venue employee. The venue may be an airport and the employee may be a TSA agent, ticket counter attendant, steward, or the like. In another example, the venue may be an employer such as a hospital, office, or workplace. In a further example, the venue may be an opera house, ballpark, concert venue, coffee shop, restaurant, or the like. Each venue may have their own group rules. In some embodiments, groups of venues may include similar or default rules (e.g., all restaurants may have a similar set of default rules). In still some embodiments, groups of similar public venues within a particular county, city, or state may utilize a different set of default rules than other groups within other counties, cities, or states.

In step 904, the user smart device 202 may unlock the health interface 100 to provide the requested information to the venue employee. In some embodiments, as described herein, the user may be required to re-verify their identity by taking another image of themselves and providing the image to the user smart device 202 and/or the TIVD system 210 for verification. In some embodiments, the user may also be required to provide a trusted document or, in some embodiments, the user smart device 202 and/or the TIVD system 210 may retrieve a trusted document from memory (or retrieve associated markers) for facial recognition. If the image is verified to be the user, the user smart device 202 may unlock the health interface 100.

In step 906, the user smart device 202 may display the health interface 100 to the venue employee. The venue employee may allow the user to attend the venue (e.g., watch a game, go to work, fly on a plane) or the like based on the infection summary and/or immunity summary.

In step 908, the venue employee may scan the code presented in the health interface 100 with their own digital device. In some embodiments, the venue employee may have their own digital device (e.g., smart phone or the like) with an application (e.g., the TIVD application 300) configured to scan the code from the health interface 100.

In step 910, the venue employee's device may provide the code and/or a venue identifier with their digital device to the TIVD system 210. The venue identifier may identify the venue, employee, or related entity. The venue employee's device may also indicate that the source of the request (e.g., network address of the venue employee's device) as well as GPS coordinates or other geolocation indicator of the venue employee's device and/or the user device the user smart device 202. In various embodiments, the venue employee's device (e.g., smart phone) include a GPS system that may provide coordinates of a location of the device. In some embodiments, the venue employee's device may provide information that may indicate location instead of or in addition to GPS coordinates. For example, the venue employee's device may provide a list of available cell towers to assist in location triangulation, a list of available Wi-Fi networks, and/or the like. The code module 414 may receive the user code from the venue employee's device. The code module 414 may also receive the venue identifier.

In step 912, the geolocation module 416 may optionally confirm that the location of the user smart device 202 is proximate to the location of the venue employee's device. In various embodiments, the /210 does not provide health information of the user without confirming that the user and the requesting venue employee are proximate to each other. In various embodiments, the code module 414 receives the code or identifier associated with the user, the user smart device 202, and/or the user's health interface 100. The geolocation module 416 may send a location request to the user the user smart device 202 (e.g., to the TIVD application 300) requesting the user's location (e.g., GPS coordinates). Upon receiving the user's GPS coordinates, the code module 414 may compare the location of the user smart device 202 coordinates to the venue employee's location. If the geolocation module 416 determines that the user smart device 202 and the venue employee are proximate to each other (e.g., within a predetermined range), the infection test verification module 404 and/or the immunity test verification module 406 may provide an infection summary or the immunity summary.

In some embodiments, the code generated by the health interface 100 and/or received by the venue employee's device may also include GPS coordinates of the user smart device 202. In this example, the venue employee's device may provide both the location of the user smart device 202 and the venue employee's device to the geolocation module 416. In this example, the geolocation module 416 may not request the location of the user smart device 202 from the user smart device 202.

In some embodiments, the TIVD application on the venue employee's device may confirm that the location of the user smart device 202 is proximate to the venue employee's device and provide the confirmation to the geolocation module 416.

In step 914, the group rules module 410 may retrieve group rules based on the requesting venue (e.g., based on the employer, restaurant, airport, or the like). Different venues may be associated with different group rules. Each set of group rules may be associated with different sets of thresholds. The group rules module 410 may retrieve group rules from the group rules datastore 422 based on a venue identifier provided by the venue employee's device. In some embodiments, the group rules module 410 may retrieve scoring criteria to generate infection scores and/or immunity scores that are particular to the venue with the user's test measurements.

In step 916, the classifier 412 may retrieve measurements from infection test information or immunity test information from a record associated with the user from the user datastore 424. The measurements may be associated with test results and previously stored in the user datastore 424. In some embodiments, the TIVD system 210 may retrieve infection test information or immunity test information from the user smart device 202 and/or any number of remote sources. The TIVD system 210 may retrieve the measurements from the infection test information or immunity test information.

In step 918, the classifier 412 may compare the measurements to a set of thresholds associated with the group rules to determine the category or classification of infection and/or immunity. In some embodiments, the immunity score module 408 may provide a score based on the measurements and the classifier 412 may compare the score to the set of thresholds. Based on the comparison, the classifier 412 may identify a classification or category.

In step 920, the classifier 412 may provide the classifications, health summary, and/or immunity summary to the health interface 100 or the venue employee's device. The classifier 412 provides the classifications, health summary, and or immunity summary to the health interface 100 or the venue employee's device if the user smart device 202 and the venue employee's device are proximate to each other.

It will be appreciated that, in some embodiments, the TIVD system 210 may confirm that the venue employee's device and/or application on the venue employee's device is authentic or authorized to receive information before providing information (e.g., by comparing an identifier of the venue employee's device to a white list, encryption key, digital certificate, and/or the like. It will be appreciated that providing the user's code may be sufficient indication that the venue employee's device is authorized to receive the information). Similarly, in some embodiments, the TIVD system 210 may confirm that the venue employee's device and/or application on the venue employee's device's request for information is authentic or authorized to receive information before providing information (e.g., by authenticating the message such as comparing a hash, identifier, encryption key, digital certificate, and/or the like).

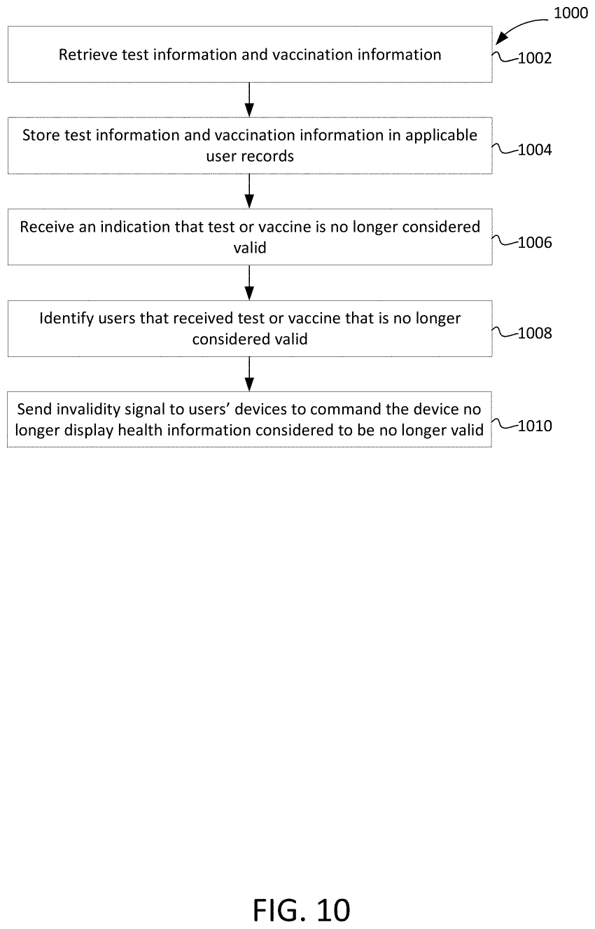

FIG. 10 is a method for tracking test results and classifications such that summaries may be controlled or otherwise indicated to be expired or no longer valid. It will be appreciated that not all tests for a particular illness may be equal. In one example, the TIVD system 210 may track the type of test, reagents used, or other information identifying the test. If test results are determined to no longer be valid, then the TIVD system 210 may send an invalidity signal to one or more user smart devices 202 to command the user smart devices 202 to not display test results. Tests and/or test results may be determined to be no longer valid for many reasons. In some embodiments, a change in regulations or health orders may result in a particular test or test results to be no longer valid or no longer considered to be trustworthy. In various embodiments, a discovery or poor testing procedure, bad reagents, or the discovery of new scientific information may result in tests and/or test results as being considered as no longer valid or untrustworthy.

The tracking module 418 may log and/or otherwise track test information regarding what tests were applied (e.g., batch number, type of test, date of test, location of the test, provider of the test, and the like). The test information may be stored in an independent database or within the appliable user's record in the user datastore 424. The infection test verification module 404 and/or the immunity score module 408 may periodically confirm that test results are still valid by contacting any number of remote sources to receive a list of invalid tests (e.g., due to failure to perform the tests under desired conditions, failures of reagents, failure of laboratory procedure, discovery that the particular test was not effective, or the like). In some embodiments, one or more remote sources may provide information regarding changes in the validity of tests or vaccines to the expiration module 420.

As discussed herein, if tests or vaccines are no longer valid, the expiration module 420 may reach out to the user smart device 202 of users who have received the tests and/or vaccines to block the health interface 100 from providing the infection and/or immunity summary or indicate that the information is no longer valid. In some embodiments, the expiration module 420 may trigger an alert to be displayed on the user smart device 202, sent by email, sent by text, or the like to notify the user of the change.

In some embodiments, test information for one or more tests may have a set expiration date when the information is no longer valid. For example, immunity based on results from an antibody test and/or vaccination may be expected to last for a predetermined period of time. The tracking module 418 may track the expiration date of the information for any number of tests and send an invalidity signal to one or more the user smart device 202 and/or indicate that information in the user record is no longer valid.

In step 1002, the tracking module 418 retrieves and logs test information and/or vaccine information that identifies information about the test and/or the vaccine. The test information and/or vaccine information may be retrieved from infection test information and immunity information (e.g., from information received by the infection test verification module 404 and/or the immunity test verification module 406).

In step 1004, the tracking module 418 may store the test information and/or vaccine information within the appropriate record(s) of the user(s).

In step 1006, the expiration module 420 receive an indication that the test or vaccine is no longer considered to be valid. In some embodiments, one or more remote sources may provide an indication to the TIVD system 210 that a test or vaccine is no longer considered valid or expired (e.g., due to failure in the delivery of the test or vaccine, ineffective performance of the test or vaccination, ineffective reagents, scientific evidence that later determined that the test or vaccine was invalid, or the validity of the test or vaccine has expired).

The indication that that the test or vaccine may be provided by any source. In various embodiments, a third-party health-related entity provides the indication. The third-party health-related entity may be any third-party health service provider, record, EPIC, HIE, independent laboratories, the FDA or any other entities.

In step 1008, the expiration module 420 may identify any number of users that may be impacted on the information that the test or vaccine they received is no longer considered valid. It will be appreciated that users all over the country and the world may receive different tests and/or vaccinations. By tracking the information of those users that utilize the TIVD system 210, the TIVD system 210 may provide updated information including indications when a test is no longer considered to be valid (e.g., the time after the test is beyond a time test threshold, there were errors associated with the testing, or the test was invalid) or the vaccine is no longer considered to be valid (e.g., the time after the vaccination is beyond a time test threshold, there were errors associated with the vaccination, or the vaccine was invalid).

In step 1010, the expiration module 420 send an invalidity signal to the users' the user smart devices 202 and/or the users' health interfaces 100 to ensure that the health interface 100 no longer depict classifications or test results related to the invalid test or vaccine. In some embodiments, after receiving the invalidity signal, the user smart device 202 may delete any infection summary, immunity summary, or other data related to the invalid test or vaccine. In some embodiments, the TIVD application 300 may display additional information on the health interface 100 to indicate that the infection summary, immunity summary, or other data related to the invalid test or vaccine are no longer considered valid.

The tracking module 418 may also track who received the user's infection summary, immunity summary, or other data related to the invalid test or vaccine. The tracking module 418 may track and store each request, when delivery of the requested information was provided, and the address or contact information of the requester (e.g., email address or identifier of a TIVD application on the requester's device). Upon determining or receiving an indication that the test or vaccine is no longer considered to be valid (and, in fact, the test or vaccine may not have been effective at the time information regarding immunity or infection was provided, the tracking module 418 may provide a message or indication that the user's past infection and immunity information is no longer valid or reliable.

It will be appreciated that a venue employee or employer may check a user's health status periodically. The duration between health status checks may be based on the dependability of the health information received from the user's health interface 100 and/or the TIVD system 210. Upon notice that the health information is no longer considered valid or is unreliable, the venue employee or employer may recheck the user's health status or require the user to take preventative action (e.g., stay away from others, stay home, take a different test, and/or take a different vaccine).

In some embodiments, the expiration module 420 may provide an alert or notification to the user indicating that the test summary, immunity summary, or other information related to their health is no longer considered valid or should no longer be considered as reliable.

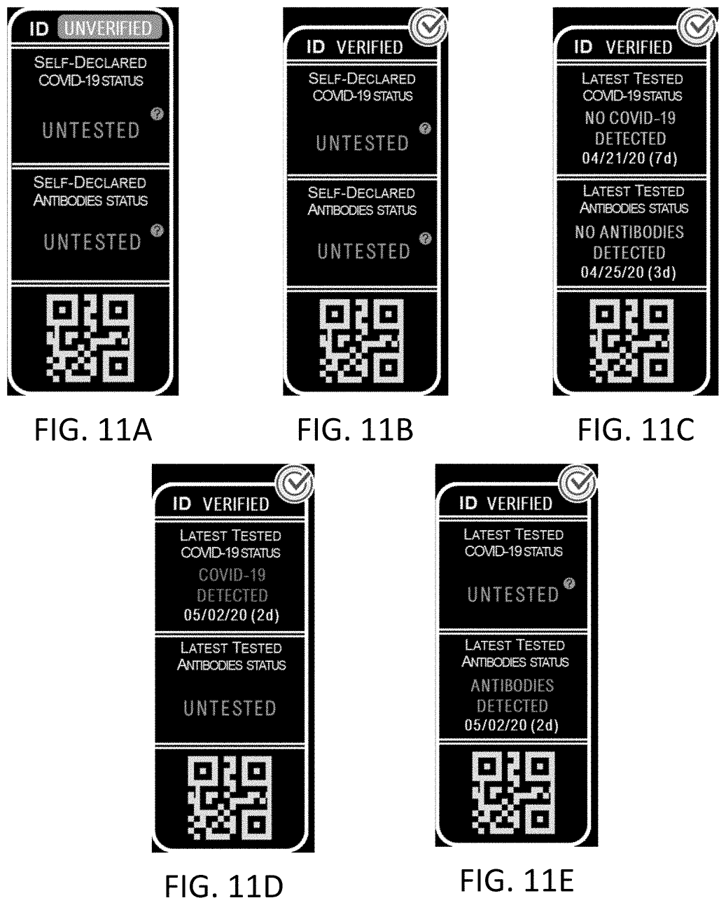

FIG. 11A depicts an example interface 100 indicating that the user's identity is unverified and the user has not been tested for infection or immunity. In some embodiments, the health interface 100 depicts the text “untested” or “unavailable” if the user's identity has not been verified.

FIG. 11B depicts an example interface 100 indicating that the user's identity is verified and the user has not been tested for infection or immunity.

FIG. 11C depicts an example interface 100 indicating that the user's identity is verified and the user has been tested for infection and immunity. The infection summary indicates that the user is uninfected with COVID-19 as of the date of the test (Apr. 21, 2020) which was seven days before the time the health interface 100 as depicted in FIG. 11C was displayed. The immunity summary indicates that no antibodies of COVID-19 were detected as of the date of the test (Apr. 25, 2020) which was three days before the time the health interface 100 as depicted in FIG. 11C was displayed

FIG. 11D depicts an example interface 100 indicating that the user's identity is verified and the user has been tested for infection but not immunity. The infection summary indicates that the user is infected with COVID-19 as of the date of the test (May 2, 2020) which was two days before the time the health interface 100 as depicted in FIG. 11D was displayed. In the interface 100, when there is an infection, the font indicating the infection may be colored red and/or other graphical elements may be used to emphasize the infection (animations, sound, blinking, different fonts, font size, and/or the like). The immunity summary indicates that the user was not tested for antibodies.

FIG. 11E depicts an example interface 100 indicating that the user's identity is verified and the user has not been tested for infection but has been tested for immunity. The infection summary indicates that the user has not been tested for infection. The immunity summary indicates that antibodies were detected as of the date of the test (May 2, 2020) which was two days before the time the health interface 100 as depicted in FIG. 11E was displayed.

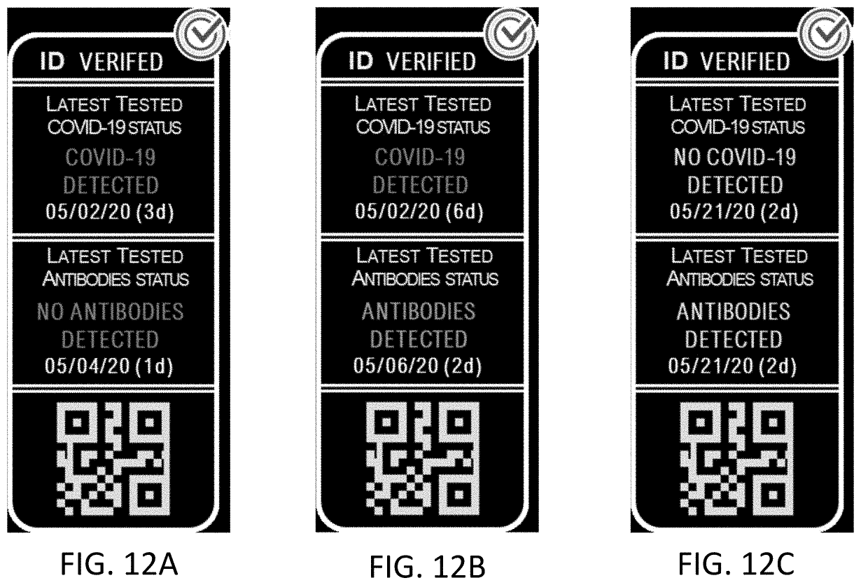

FIG. 12A depicts an example interface 100 indicating that the user's identity is verified and the user has been tested for infection and immunity. The infection summary indicates that the user is infected with COVID-19 as of the date of the test (May 2, 2020) which was three days before the time the health interface 100 as depicted in FIG. 12A. The immunity summary indicates that no antibodies were detected as of the date of the test (May 4, 2020) which was one day before the time the health interface 100 as depicted in FIG. 12A was displayed.

FIG. 12B depicts an example interface 100 indicating that the user's identity is verified and the user has been tested for infection and immunity. The infection summary indicates that the user is infected with COVID-19 as of the date of the test (May 2, 2020) which was six days before the time the health interface 100 as depicted in FIG. 12B. The immunity summary indicates that antibodies were detected as of the date of the test (May 6, 2020) which was two days before the time the health interface 100 as depicted in FIG. 12B was displayed.

FIG. 12C depicts an example interface 100 indicating that the user's identity is verified and the user has been tested for infection and immunity. The infection summary indicates that the user is not infected with COVID-19 as of the date of the test (May 21, 2020) which was two days before the time the health interface 100 as depicted in FIG. 12C. The immunity summary indicates that antibodies were detected as of the date of the test (May 21, 2020) which was two days before the time the health interface 100 as depicted in FIG. 12C was displayed.

FIG. 13 depicts a block diagram of an example digital device 1302 according to some embodiments. The digital device 1302 comprises a processor 1304, a memory 1306, a storage 1308, an input device 1310, a communication network interface 1312 and an output device 1314. Processor 1304 is configured to execute executable instructions (e.g., programs). In some embodiments, the processor 1304 comprises circuitry or any processor capable of processing the executable instructions.

Memory 1306 stores data. Some examples of memory 1306 include storage devices, such as RAM, ROM, RAM cache, virtual memory, etc. In various embodiments, working data is stored within memory 1306. The data within memory 1306 may be cleared or ultimately transferred to storage 1308.

Storage 1308 includes any storage configured to retrieve and store data. Some examples of storage 1308 includes flash drives, hard drives, optical drives, and/or magnetic tape. Each of memory system 1306 and storage system 1308 comprises a computer-readable medium, which stores instructions or programs executable by processor 1304. The storage 1308 may include nontransitory computer readable media.

Input device 1310 is any device that inputs data (e.g., mouse, keyboard, stylus). Output device 1314 outputs data (e.g., speaker, display, virtual reality headset). It will be appreciated that storage 1308, input device 1310 and output device 1314 may be optional. For example, routers/switchers may comprise processor 1304 and memory 1306 as well as a device to receive and output data (e.g., communication network interface 1312 and/or output device 1314).

Communication network interface 1312 may be coupled to a network (e.g. communication network 214) via communication network interface 1312. Communication network interface 1312 may support communication over an Ethernet connection, a serial connection, a parallel connection, and/or an ATA connection. Communication network interface 1312 may also support wireless communication (e.g., 802.11 a/b/g/n, WiMax, LTE, WiFi). It will be apparent that communication network interface 1312 may support many wired and wireless standards.

The output device 1314 outputs data (e.g., speaker, display, virtual reality headset). It will be appreciated that the storage 1308, input device 1310 and output device 1314 may be optional. For example, routers/switchers may comprise processor 1304 and memory 1306 as well as a device to receive and output data (e.g., communication network interface 1312 and/or output device 1314).

It should be understood that although not shown, other hardware and/or software components could be used in conjunction with the digital device 1300. Examples include, but are not limited to: microcode, device drivers, redundant processing units, and external disk drive arrays, RAID systems, tape drives, and data archival storage systems, etc.

Exemplary embodiments are described herein in detail with reference to the accompanying drawings. However, the present disclosure can be implemented in various manners, and thus should not be construed to be limited to the embodiments disclosed herein. On the contrary, those embodiments are provided for the thorough and complete understanding of the present disclosure, and completely conveying the scope of the present disclosure to those skilled in the art.

As will be appreciated by one skilled in the art, aspects of one or more embodiments may be embodied as a system, method or computer program product. Accordingly, aspects may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband/or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects discussed herein may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of some of the embodiments are described herein with reference to flowchart illustrations and/or block diagrams of methods, apparatus (systems) and computer program products according to embodiments. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a nontransitory computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components. These and other variations, modifications, additions, and improvements fall within the scope of the subject matter herein.

It may be apparent to those skilled in the art that various modifications may be made and other embodiments may be used without departing from the broader scope of the discussion herein. Therefore, these and other variations upon the example embodiments are intended to be covered by the disclosure herein.

1. A nontransitory computer readable medium comprising instructions executable by a processor, the instructions being executable to perform a method, the method comprising:
receiving, from a user device, a user identifier, the user identifier being associated with a user of the user device;
confirming that the user's identity has been confirmed;
retrieving, from a remote health record maintained by a third-party health service provider, health information regarding a virus-related test provided on the user, the virus-related test including a test identifier, a date when the virus-related test was given, and virus-related test results;
providing a virus-related test status based on the virus-related test results to the user device;
if the virus-related test is determined to be invalid, receiving an invalidity indication from a third-party health-related entity, the invalidity indication indicating that a particular test is no longer considered valid;
reviewing a record of the user of the user device to confirm if the user received the particular test;
determining that the virus-related test is no longer valid based on the invalidity indication;
providing a notice to the user device that the virus-related test status is no longer considered to be valid; and
providing an update to the virus-related test status based on the invalidity indication.

2. The nontransitory computer readable medium of claim 1, wherein the virus-related test is an infection test and the virus-related test status is an indication if the user is infected.

3. The nontransitory computer readable medium of claim 1, wherein the virus-related test is an immunity test and the virus-related test status is an indication if the user is immune.

4. The nontransitory computer readable medium of claim 1, the method further comprising:
receiving, from an other user device, an other user identifier, the user device and the other user device being remote from each other;
confirming that the other user's identity has been confirmed;
retrieving, from an other remote health record maintained by an other third-party health service provider, health information regarding an other virus-related test provided on the other user, the other virus-related test including an other test identifier, an other date when the other virus-related test was given, and other virus-related test results, the third-party health service provider and the other third-party health service provider being remote to each other and being operated by different entities; and
providing an other virus-related test status based on the other virus-related test results to the other user device.

5. The nontransitory computer readable medium of claim 1, further comprising reviewing the record of the other user to confirm if the other user received the particular test and providing a notice to the other user device that the virus-related test status is no longer considered to be valid.

6. The nontransitory computer readable medium of claim 5, further comprising scoring the virus-related test results, wherein the virus-related test status is based on the scoring.

7. The nontransitory computer readable medium of claim 5, further comprising receiving group rules associated with a venue and comparing the virus-related test results to at least one threshold of the group rules, wherein the virus-related test status is based on the comparison.

8. The nontransitory computer readable medium of claim 1, wherein receiving, from the user device, comprises receiving a user image from the user device, the method further comprising receiving information regarding a trusted image of the user and performing identity confirmation based on the user image and the information regarding the trusted image to confirm the user's identity.

9. The nontransitory computer readable medium of claim 1, wherein retrieving, from the remote health record maintained by the third-party health service provider, health information regarding the virus-related test provided on the user comprises retrieving an encryption key associated with the user and providing a request for the health information with the encryption key to the third-party health service provider.

10. The nontransitory computer readable medium of claim 9, the method further comprising receiving a virus-related test indication associated with the user, the virus-related test indication indicating the third-party health service provider as having the health information regarding the virus-related test provided on the user.

11. The nontransitory computer readable medium of claim 9, wherein retrieving, from the remote health record maintained by the third-party health service provider, health information regarding the virus-related test provided on the user comprises providing a request for the health information using an identifier that does not refer to the user.

12. The nontransitory computer readable medium of claim 1, wherein retrieving, from the remote health record maintained by the third-party health service provider, health information regarding the virus-related test provided on the user comprises receiving, without providing an initial request, the health information regarding the virus-related test from the third-party health service provider, storing the health information regarding the virus-related test, and retrieving the health information regarding the virus-related test from storage.

13. The nontransitory computer readable medium of claim 1, the method further comprising:
receiving, from the user device, the user identifier;
confirming that the user's identity has been confirmed;
retrieving, from a remote health record maintained by a third-party health service provider, health information regarding a vaccination provided to the user; and
providing an immunity classification to the user device, the immunity classification indicating that the user is immune to at least one illness.

14. The nontransitory computer readable medium of claim 1, the method further comprising:
receiving, from the user device, the user identifier;
confirming that the user's identity has been confirmed;
retrieving, from a remote health record maintained by a third-party health service provider, health information regarding an antibody test provided on the user and immunity test results;
scoring the immunity test results to classify the user regarding possible immunity to infection to at least on illness; and
providing a immunity classification based on the scoring to the user device, the immunity classification indicating whether the user is immune or likely immune.

15. A method comprising:
receiving, from a user device, a user identifier, the user identifier being associated with a user of the user device;
confirming that the user's identity has been confirmed;
retrieving, from a remote health record maintained by a third-party health service provider, health information regarding a virus-related test provided on the user, the virus-related test including a test identifier, a date when the virus-related test was given, and virus-related test results;
providing a virus-related test status based on the virus-related test results to the user device;
if the virus-related test is determined to be invalid, receiving an invalidity indication from a third-party health-related entity, the invalidity indication indicating that a particular test is no longer considered valid;
reviewing a record of the user of the user device to confirm if the user received the particular test;
determining that the virus-related test is no longer valid based on the invalidity indication;
providing a notice to the user device that the virus-related test status is no longer considered to be valid; and
providing an update to the virus-related test status based on the invalidity indication.

16. The method of claim 15, wherein the virus-related test is an infection test and the virus-related test status is an indication if the user is infected.

17. The method of claim 15, wherein the virus-related test is an immunity test and the virus-related test status is an indication if the user is immune.

18. The method of claim 15, further comprising:
receiving, from an other user device, an other user identifier, the user device and the other user device being remote from each other;
confirming that the other user's identity has been confirmed;
retrieving, from an other remote health record maintained by an other third-party health service provider, health information regarding an other virus-related test provided on the other user, the other virus-related test including an other test identifier, an other date when the other virus-related test was given, and other virus-related test results, the third-party health service provider and the other third-party health service provider being remote to each other and being operated by different entities; and
providing an other virus-related test status based on the other virus-related test results to the other user device.

19. The method of claim 18, further comprising reviewing the record of the other user to confirm if the other user received the particular test and providing a notice to the other user device that the virus-related test status is no longer considered to be valid.

20. The method of claim 15, further comprising scoring the virus-related test results, wherein the virus-related test status is based on the scoring.

21. The method of claim 15, further comprising receiving group rules associated with a venue and comparing the virus-related test results to at least one threshold of the group rules, wherein the virus-related test status is based on the comparison.

22. The method of claim 15, wherein receiving, from the user device, comprises receiving a user image from the user device, the method further comprising receiving information regarding a trusted image of the user and performing identity confirmation based on the user image and the information regarding the trusted image to confirm the user's identity.

23. The method of claim 15, wherein retrieving, from the remote health record maintained by the third-party health service provider, health information regarding the virus-related test provided on the user comprises retrieving an encryption key associated with the user and providing a request for the health information with the encryption key to the third-party health service provider.

24. The method of claim 23, the method further comprising receiving a virus-related test indication associated with the user, the virus-related test indication indicating the third-party health service provider as having the health information regarding the virus-related test provided on the user.

25. The method of claim 23, wherein retrieving, from the remote health record maintained by the third-party health service provider, health information regarding the virus-related test provided on the user comprises providing a request for the health information using an identifier that does not refer to the user.

26. The method of claim 15, wherein retrieving, from the remote health record maintained by the third-party health service provider, health information regarding the virus-related test provided on the user comprises receiving, without providing an initial request, the health information regarding the virus-related test from the third-party health service provider, storing the health information regarding the virus-related test, and retrieving the health information regarding the virus-related test from storage.

27. The method of claim 15, the method further comprising:
receiving, from the user device, the user identifier;
confirming that the user's identity has been confirmed;
retrieving, from a remote health record maintained by a third-party health service provider, health information regarding a vaccination provided to the user; and
providing an immunity classification to the user device, the immunity classification indicating that the user is immune to at least one illness.

28. The method claim 15, the method further comprising:
receiving, from the user device, the user identifier;
confirming that the user's identity has been confirmed;
retrieving, from a remote health record maintained by a third-party health service provider, health information regarding an antibody test provided on the user and immunity test results;
scoring the immunity test results to classify the user regarding possible immunity to infection to at least on illness; and
providing a immunity classification based on the scoring to the user device, the immunity classification indicating whether the user is immune or likely immune.

29. A system comprising:
at least one processor;
memory containing instructions to control the at least one processor to:
receive, from a user device, a user identifier, the user identifier being associated with a user of the user device;
confirm that the user's identity has been confirmed;
retrieve, from a remote health record maintained by a third-party health service provider, health information regarding a virus-related test provided on the user, the virus-related test including a test identifier, a date when the virus-related test was given, and virus-related test results;
provide a virus-related test status based on the virus-related test results to the user device;
if the virus-related test is determined to be invalid, receive an invalidity indication from a third-party health-related entity, the invalidity indication indicating that a particular test is no longer considered valid;
review a record of the user of the user device to confirm if the user received the particular test;
determine that the virus-related test is no longer valid based on the invalidity indication;
provide a notice to the user device that the virus-related test status is no longer considered to be valid; and
provide an update to the virus-related test status based on the invalidity indication.
